(19) United States
(12) Patent Application Publication
(10) Pub. No.: US 2024/0139173 A1
(43) Pub. Date: May 2, 2024

(54) OPHTHALMIC FORMULATIONS AND USES THEREOF

(71) Applicant: Aldeyra Therapeutics, Inc., Lexington, MA (US)

(72) Inventors: David CLARK, Winchester, MA (US); Susan MACDONALD, Danvers, MA (US); Stephen Gitu MACHATHA, Wilmington, MA (US); Lise Lund KJEMS, Concord, MA (US)

(21) Appl. No.: 18/367,324

(22) Filed: Sep. 12, 2023

Related U.S. Application Data
(63) Continuation of application No. 16/825,898, filed on Mar. 20, 2020, now Pat. No. 11,786,518.
(60) Provisional application No. 62/857,874, filed on Jun. 6, 2019.
(60) Provisional application No. 62/824,233, filed on Mar. 26, 2019.

(51) Int. Cl. A61K 31/47; A61K 9/00; A61K 9/08; A61K 47/40; A61P 27/14

(52) U.S. Cl. CPC A61K 31/47; A61K 9/0048; A61K 9/08; A61K 47/40; A61P 27/14

(57) ABSTRACT

The present invention provides a reproxalap ophthalmic solution, and methods of using the same for treating a disease or disorder such as allergic conjunctivitis.

### CROSS-REFERENCE TO RELATED APPLICATIONS

This application claims the benefit under 35 U.S.C. § 119(e) of U.S. provisional application No. 62/824,233, filed Mar. 26, 2019, and U.S. provisional application No. 62/857,874, filed Jun. 6, 2019, the contents of each of which is incorporated by reference herein in their entireties.

### TECHNICAL FIELD

The present invention relates to ophthalmic solutions comprising reproxalap, or a pharmaceutically acceptable salt thereof, for treatment of a disease or disorder such as allergic conjunctivitis (AC).

### BACKGROUND

Acute allergic conjunctivitis (AC) is experienced by approximately 40% of the general population (Singh et al., J Allergy Clin Immunol., 2010, 126(4):778-783). AC is associated with Type I (immediate) hypersensitivity reactions, which involve immunoglobulin E (IgE)-mediated release of histamine and other mediators from mast cells and basophils. Mast cell degranulation leads to release of inflammatory mediators and activation of enzymatic cascades generating pro-inflammatory mediators (Mishra et al., Recent Pat Inflamm Allergy Drug Discov., 2011, 5(1):26-36). In the eye, release of a variety of mediators leads to inflammation of the conjunctival mucosa that also affects the cornea and eyelids, resulting in symptoms that include itching and burning, tearing, chemosis (conjunctival edema), conjunctival injection, hyperemia, eyelid edema, and mucus discharge.

The early symptoms of allergic conjunctivitis, occurring within minutes of allergen exposure, are likely due primarily to the release of histamine (Leonardi, A., Exp Eye Res. 2013, 117:106-17), which peaks in tears about five minutes after exposure to allergen and significantly diminishes over 30 to 40 minutes (Ackerman et al., Ther Adv Chronic Dis., 2016, 7(1):52-67.2016). Immediately following the rapid rise and fall of histamine, the symptoms of allergic conjunctivitis are perpetuated by non-histaminic inflammatory mediators, such as cellular infiltrate, cytokines, leukotrienes, proteases, and other factors (Leonardi, A., Exp Eye Res., 2013, 117:106-17), including aldehydes. Aldehydes are pro-inflammatory mediators of both allergic (Th2) and autoimmune (Th1) diseases, and elevated levels of toxic aldehydes are associated with allergic conjunctivitis and other ocular and systemic diseases (Bacsi et al., J Allergy Clin Immunol., 2005, 116(4): 836-843; Boldogh et al., J Clin Invest., 2005, 115(8): 2169-2179; Wakamatsu et al., Mol Vis., 2010, 16: 2465-2475).

Aldehydes are reactive and bind to proteins, carbohydrates, lipids and nucleic acids (Esterbauer et al., Free Radical Biology and Medicine, 1990, 11(1):81-128). Free aldehydes—aldehydes not sequestered or otherwise protected in specific metabolic processes—can be toxic, and aldehyde binding to cellular constituents can lead to inflammation (Yadav et al., Oxidative Medicine and Cellular Longevity 2013, Volume 2013, Article ID 690545), molecular dysfunction (O'Brien et al., Critical Reviews in Toxicology, 2005, 35(7):609-62), and the accumulation of indigestible metabolites, such as lipofuscin components in the retina (Boyer et al., J Biol Chem., 2012, 287:22276-86).

In biological systems, aldehydes are formed by a variety of processes, including the oxidation of alcohols, polyamine and glucose metabolism, and oxidative stress. In some disease states, aldehyde concentrations may be increased. Increases in aldehyde concentrations, particularly malonyldialdehyde (MDA), which is thought to be most commonly derived from lipid peroxidation, has been described in a variety of inflammatory ocular diseases, including pterygium, Behcet's Disease, Sjögren's Syndrome, anterior uveitis, and dry eye disease (Sandikci et al., Acta Dermato-Venereologica, 2003, 83(5):342-6; Cejkova et al., Histology and Histopathology, 2007, 22(9):997-1003; Balci et al., Molecular Vision, 2011, 17:443-7; Turk et al., Ocular Immunology and Inflammation, 2014, 22(2):127-32; Choi et al., Current Eye Research, 2016, 41(9):1143-9).

Thus, there is an unmet need for improved ophthalmic formulations of drugs capable of treating allergic conjunctivitis.

### SUMMARY

In one aspect, the present invention relates to an ophthalmic solution comprising reproxalap, or a pharmaceutically acceptable salt thereof, and a pharmaceutically acceptable excipient, wherein the concentration of reproxalap, or a pharmaceutically acceptable salt thereof, is about 0.5% w/v or less and about 0.1% w/v or greater. In some embodiments, the reproxalap and a cyclodextrin excipient in the ophthalmic solution in a less than 1:2.1 molar ratio. In some embodiments, concentration of reproxalap, or a pharmaceutically acceptable salt thereof, is about 0.2% w/v to about 0.3% w/v.

In some embodiments, the ophthalmic solution comprises about 0.25% w/v reproxalap and a pharmaceutically acceptable excipient selected from a cyclodextrin. In some embodiments, the cyclodextrin is at about 7% w/v. In some embodiments, the cyclodextrin is at about 11% w/v. In some embodiments, the cyclodextrin is sulfobutylether-β-cyclodextrin or hydroxypropyl-β-cyclodextrin.

In some embodiments, the ophthalmic solution comprises about 0.5% w/v reproxalap and a pharmaceutically acceptable excipient selected from a cyclodextrin. In some embodiments, the cyclodextrin is at about 9.5% w/v. In some embodiments, the cyclodextrin is sulfobutylether-β-cyclodextrin or hydroxypropyl-β-cyclodextrin.

In another aspect, the present invention relates to a method for treating allergic conjunctivitis in a subject, comprising topically administering to any eye of a subject in need thereof a therapeutically effective amount of an ophthalmic solution of the invention. In some embodiments, the allergic conjunctivitis treated is seasonal allergic conjunctivitis. In some embodiments, the allergic conjunctivitis treated is contact conjunctivitis. In some embodiments, the allergic conjunctivitis treated is perennial conjunctivitis.

In some embodiments of the method, an ophthalmic solution of the invention is administered prior to exposure of the subject to an eye allergen. In some embodiments, the ophthalmic solution is administered to an eye of a subject in need thereof in the time period for durability of the therapeutic effect, e.g., for a 0.25% w/v or 0.5% w/v reproxalap solution, prior to an expected or certain exposure to an eye allergen. In some embodiments, the ophthalmic solution is administered 3.5 h or less, 3 h or less, 2.5 h or less, 2 h or less, 1.5 h or less, 1 h or less, 0.5 h or less, 25 min or less, 20 min or less, 25 min or less, 20 min or less, 15 min or less, 10 min or less, or 5 min or less before an expected or certain exposure to an eye allergen, or immediately prior to an expected or certain exposure to an eye allergen.

In some embodiments, the subject for treatment prior to an expected or certain exposure to an eye allergen has a history of allergic conjunctivitis. In some embodiments, the subject for treatment prior to an expected or certain exposure to an eye allergen has a history of allergic conjunctivitis and has a positive allergen skin test for one or more eye allergens. In some embodiments, the subject for treatment prior to an expected or certain exposure to an eye allergen has been previously clinically diagnosed with allergic conjunctivitis. In some embodiments, the subject for treatment prior to an expected or certain exposure to an eye allergen has been previously clinically diagnosed with allergic conjunctivitis, and has a positive allergen skin test for one or more eye allergens.

In some embodiments of the method, an ophthalmic solution of the invention is administered at or after exposure to an eye allergen. In some embodiments, the ophthalmic solution of the invention is administered immediately after exposure to an eye allergen. In some embodiments, the ophthalmic solution of the invention is administered at or after the onset of symptoms of allergic conjunctivitis. In some embodiments, a method for treating allergic conjunctivitis in a subject comprises topically administering to an eye of a subject in need thereof an ophthalmic solution of the invention at or after exposure to an eye allergen, or at or after onset of symptoms of allergic conjunctivitis, where the ophthalmic solution is administered six times a day, five times a day, four times a day (QID), three times a day (TID), two times a day (BID), once a day (QD), or as needed (PRN). In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where the ophthalmic solution is administered six times a day, five times a day, four times a day (QID), three times a day (TID), two times a day (BID), once a day (QD), followed by administration as needed (PRN). In some embodiments of the method, an ophthalmic solution of the invention is administered four times a day (QID) followed by administration as needed (PRN).

In some embodiments, the treatment at or after exposure to an eye allergen or at or after the onset of symptoms of allergic conjunctivitis can be further divided into treatment in an initiation phase, exacerbation phase, and/or maintenance phase. In some embodiments, the ophthalmic solution is administered six times a day, five times a day, or four times a day (QID) in the initiation phase and/or exacerbation phase, followed by two times a day (BID), once a day (QD) or as needed (PRN) in the maintenance phase.

In some embodiments, a method for treating allergic conjunctivitis comprises administering to an eye of a subject in need thereof an ophthalmic solution of the invention prior to an expected or certain exposure to an eye allergen; and further comprising administering an ophthalmic solution of the invention to the eye of the subject at or after exposure to the eye allergen, or at or after onset of symptoms of allergic conjunctivitis. In some embodiments, a method of treating allergic conjunctivitis comprises a first treatment phase and a second treatment phase, wherein the first treatment phase comprises administering an ophthalmic solution of the invention to an eye of a subject in need thereof prior to an expected or certain exposure to an eye allergen, or immediately prior to expected or certain exposure to an eye allergen, and the second treatment phrase comprises administering an ophthalmic solution of the invention to the eye at or after exposure to an eye allergen, or at or after the onset of symptoms of allergic conjunctivitis.

In some embodiments, in the first treatment phase of this two phase treatment regimen, an ophthalmic solution of the invention is administered 3.5 h or less, 3 h or less, 2.5 h or less, 2 h or less, 1.5 h or less, 1 h or less, 0.5 h or less, 25 min or less, 20 min or less, 25 min or less, 20 min or less, 15 min or less, 10 min or less, or 5 min or less prior to an expected or certain exposure to an eye allergen, or immediately prior to expected or certain exposure to an eye allergen.

In some embodiments, in the second treatment phase, an ophthalmic solution of the invention is administered within 5 min, 10 min, 15 min, 20 min, 25 min, 30 min, 35 min, 40 min, 45 min, 50 min, 60 min (1 h), 75 min (1.25 h), 90 min (1.5 h), 105 min (1.75 h), or 120 min (2 h) of exposure to an eye allergen or after the onset of symptoms of allergic conjunctivitis. In some embodiments, in the second treatment phase, an ophthalmic solution of the invention is administered six times a day, five times a day, four times a day (QID), three times a day (TID), two times a day (BID), once a day (QD) or as needed (PRN). In some embodiments, in the second treatment phase, an ophthalmic solution of the invention is administered six times a day, five times a day, four times a day (QID), three times a day (TID), two times a day (BID), once a day (QD), followed by administration as needed (PRN). In some embodiments, in the second treatment phase, an ophthalmic solution of the invention is administered four times a day (QID) followed by administration as needed (PRN).

In some embodiments, the second treatment phase can be further divided into treatment in an initiation phase, exacerbation phase, and/or maintenance phase. In some embodiments, the ophthalmic solution is administered six times a day, five times a day, or four times a day (QID) in the initiation phase and/or exacerbation phase, followed by two times a day (BID), once a day (QD) or as needed (PRN) in the maintenance phase.

### BRIEF DESCRIPTION OF THE DRAWINGS

FIG. 1 depicts area under the curve calculations (AUC) for Ocular Itch Score (0-4) 10 to 60 minutes after allergen challenge. SEM=Standard error of the mean; Ocular Itch Scale 0 (no itch) to 4 (incapacitating itch).

FIG. 2 depicts the probability of a two-point response in ocular itch score over time after an allergen challenge. For both concentrations of reproxalap, the results were statistically superior to vehicle in achieving the result. p=0.0005 for 0.25% reproxalap; p=0.0169 for 0.5% reproxalap.

FIG. 3 depicts the probability of an ocular itch score of 0 (therapeutic cure) over time after an allergen challenge. For both concentrations of reproxalap, the results were statistically superior to vehicle in achieving complete reduction of ocular itch. p=0.0006 for 0.25% reproxalap; p=0.0045 for 0.5% reproxalap.

FIG. 4 depicts ocular itch score (0-100) during 200 minutes of allergen exposure in a group chamber model of allergic conjunctivitis. The results are shown for 0.25% w/v Reproxalap administered at 5 min prior to and 95 min after exposure to an allergen (ragweed). Treatment with Reproxalap shows statistically significant reduction in ocular itch vs. vehicle for more than 3 hrs of exposure to the allergen.

FIG. 5 depicts ocular redness score (0-4) during 200 minutes of allergen exposure in a group chamber model of allergic conjunctivitis. The results are shown for 0.25% w/v Reproxalap administered at 5 min prior to and 95 min after exposure to allergen (ragweed). Treatment with Reproxalap shows statistically significant reduction in ocular redness vs. vehicle for more than 3 hrs of exposure to allergen.

### DETAILED DESCRIPTION

### 1. General Description of Certain Embodiments

Reproxalap topical ocular solution is being developed for treatment of ocular inflammation. The drug product, in various strengths, has completed a Phase 1 clinical trial, as well as a controlled, double-masked Phase 2a clinical trial in allergic conjunctivitis, a controlled, double-masked Phase 2b clinical trial in allergic conjunctivitis, a controlled, double-masked Phase 2 clinical trial in noninfectious anterior uveitis, a Phase 2a clinical trial in dry eye disease, and a controlled, double-masked Phase 2b clinical trial in dry eye disease. The maximum exposure to reproxalap in these completed clinical trials has been dosing with 0.5% w/v strength for six weeks, for treating subjects with noninfectious anterior uveitis.

It has now been found that certain topical (ophthalmic) formulations (e.g., solutions) of reproxalap are surprisingly effective in treating allergic conjunctivitis. Without wishing to be bound by theory, it is believed that certain concentrations of reproxalap in combination with excipients and/or ratios of reproxalap to excipients exhibit increased efficacy over previous ophthalmic solutions of reproxalap. Furthermore, in some embodiments, the presently disclosed topical formulations result in reduced instillation site pain and irritation in human patients relative to previously known ophthalmic solutions of reproxalap. In some embodiments, reduced instillation site pain and irritation leads to increased patient compliance and improved outcomes.

In some embodiments, the presently disclosed ophthalmic solutions exhibit an improved (e.g., extended) release of reproxalap in comparison with previously known reproxalap ophthalmic solutions. Without wishing to be bound by theory, it is believed that the presently disclosed ophthalmic solutions may provide extended or delayed release of reproxalap due to interactions of reproxalap with the excipient(s). In some embodiments, particular ratios of reproxalap:excipient(s) in the presently disclosed ophthalmic solutions exhibit increased efficacy.

In some embodiments, after administration to a patient population, an ophthalmic solution of the present disclosure provides a statistically significant reduction in the ocular itch scale of a patient population suffering from allergic conjunctivitis. In some embodiments, after administration to a patient population, an ophthalmic solution of the present disclosure provides a statistically significant (p<0.05) reduction by at least 1 point in the ocular itch scale of a patient population suffering from allergic conjunctivitis. In some embodiments, after administration to a patient population, an ophthalmic solution of the present disclosure provides a statistically significant (p<0.05) reduction by at least 2 points in the ocular itch scale of a patient population suffering from allergic conjunctivitis. Ocular Itch Scale 0 (no itch) to 4 (incapacitating itch).

In some embodiments, the present invention provides an ophthalmic solution comprising reproxalap, or a pharmaceutically acceptable salt thereof, and a pharmaceutically acceptable excipient, wherein the concentration of reproxalap, or a pharmaceutically acceptable salt thereof, is about 0.5% w/v or less and 0.1% w/v or greater. In some embodiments, the present invention provides a method for treating allergic conjunctivitis in a subject, comprising topically administering an ophthalmic solution described herein to the subject in need thereof.

In some embodiments, a disclosed ophthalmic solution comprises reproxalap and a cyclodextrin excipient in a less than 1:2.1 molar ratio. In some embodiments, the ratio is a 1:2.1 to about 1:25 ratio. In some embodiments, the ratio is about 1:2.2 to 1:20, 1:2.5 to 1:20, 1:2.5 to 1:10, 1:2.75 to 1:10, 1:3 to 1:8, 1:3.5 to 1:7, 1:4 to 1:6, or 1:4 to 1:5. In some embodiments, the ratio is about 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4.0, 1:4.1, 1:4.2, 1:4.3, 1:4.4, 1:4.5, 1:4.6, 1:4.7, 1:4.8, 1:4.9, 1:5.0, 1:5.1, 1:5.2, 1:5.3, 1:5.4, 1:5.5, 1:6, 1:7, 1:8, 1:9, 1:10, 1:12, 1:15, 1:20, or 1:25. In some embodiments, the ratio is one of those in Table 1, below, e.g. a ratio in Table 1+/−10%. In some embodiments, the cyclodextrin is sulfobutylether β-cyclodextrin (SBECD). In some embodiments, the cyclodextrin is hydroxypropyl-β-cyclodextrin (2-hydroxypropyl-β-cyclodextrin: HPCD).

In some embodiments, the cyclodextrin excipient is one of those described herein, such as sulfobutylether β-cyclodextrin (SBECD).

TABLE 1
Reproxalap Ophthalmic Solutions

| Formulation | | | SBECD | reproxalap | Molar ratio | |
|---|---|---|---|---|---|---|
| (reproxalap/ | reproxalap | SBECD | * (MW = | (MW = | (SBECD/ | |
| SBECD, % w/v) | (g/L) | (g/L) | 2163 g/mole) | 236.7 g/mole) | Reproxalap) | |
| 0.25/7 | 2.5 | 70 | 0.032 | 0.011 | 3.06 | ~3 moles of |
| | | | | | | SBECD per |
| | | | | | | mole of API |
| 0.5/9.5 | 5 | 95 | 0.044 | 0.021 | 2.08 | ~2 moles of |
| | | | | | | SBECD per |
| | | | | | | mole of API |
| 0.25/11 | 2.5 | 110 | 0.051 | 0.011 | 4.81 | ~5 moles of |
| | | | | | | SBECD per |
| | | | | | | mole of API |

* average degree of substitution = 6.5.

In some embodiments, the ophthalmic solution comprises about 0.2% to 0.4% reproxalap and about 7% to 25% w/v of a cyclodextrin excipient, such as SBECD or HPCD. In some embodiments, the ophthalmic solution comprises about 0.2%, 0.25%, 0.3%, 0.35%, or 0.4% reproxalap and about 7% to 25% w/v of a cyclodextrin excipient, such as SBECD or HPCD.

In some embodiments, the ophthalmic solution comprises about 0.25% reproxalap and about 4.7% to about 25% w/v of a cyclodextrin excipient such as SBECD or HPCD.

In some embodiments, the ophthalmic solution comprises about 0.25% reproxalap and about 7% to 25% w/v of a cyclodextrin excipient such as SBECD or HPCD.

In some embodiments, the ophthalmic solution comprises about 0.25% reproxalap and about 4.75% to about 11% w/v of a cyclodextrin excipient such as SBECD or HPCD.

In some embodiments, the ophthalmic solution comprises about 0.5% reproxalap and about 9.5% to about 11% w/v of a cyclodextrin excipient such as SBECD or HPCD.

In some embodiments, the ophthalmic solution comprises about 0.25% reproxalap and about 7% w/v of a cyclodextrin excipient such as SBECD or HPCD.

In some embodiments, the ophthalmic solution comprises about 0.25% reproxalap and about 11% w/v of a cyclodextrin excipient such as SBECD or HPCD.

### 2. Definitions

The general terms used herein are defined with the following meanings, unless explicitly stated otherwise.

The term “comprising” and “including” are used herein in their open-ended and non-limiting sense unless otherwise noted.

The terms “a” and “an” and “the” and similar references in the context of describing the invention (especially in the context of the following claims) are to be construed to cover both the singular and the plural, unless otherwise indicated herein or clearly contradicted by context. Where the plural form is used for compounds, salts, and the like, this is taken to mean also a single compound, salt, or the like.

The term “pharmaceutically acceptable” is defined herein to refer to those compounds, biologic agents, materials, compositions and/or dosage forms, which are, within the scope of sound medical judgment, suitable for contact with the tissues a subject e.g., a mammal or human, without excessive toxicity, irritation allergic response and other problem complications commensurate with a reasonable benefit/risk ratio.

The term “treating” or “treatment” as used herein comprises a treatment relieving, reducing or alleviating at least one symptom in a subject or affecting a delay of progression of a disease, condition and/or disorder. For example, treatment can be the diminishment of one or several signs or symptoms of a disorder or complete eradication of a disorder. Within the meaning of the present invention, the term “treat” also denotes to arrest, delay the onset of (e.g., the period prior to clinical manifestation of a disease) and/or reduce the risk of developing or worsening a disease.

The term “subject” or “patient” as used herein includes animals, such as mammals, e.g., humans, dogs, cows, horses, pigs, sheep, goats, cats, mice, rabbits, rats and transgenic non-human animals. In some embodiments, the subject is a human.

The term “about” or “approximately” shall have the meaning of within 10% of a given value or range. In some embodiments, the term “about” refers to within 10%, 9%, 8%, 7%, 6%, 5%, 4%, 3%, 2%, or 1% of a given value.

The term “w/v” as used herein refers to “gram/mL” (weight over volume), which is a concentration unit. For example, 7% w/v is equivalent to 70 mg/mL.

Reproxalap has the following structure:

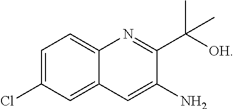 [SMILES: CC(C)(O)c1nc2ccc(Cl)cc2cc1N]

Without wishing to be bound by any particular theory, reproxalap functions as an aldehyde sequestering agent, or “trap,” which binds rapidly to aldehydes and forms a cyclic product.

### 3. Ophthalmic Solutions

An ophthalmic solution of the invention comprises reproxalap, or a pharmaceutically acceptable salt thereof, at a concentration suitable for effectively treating allergic conjunctivitis, in particular without causing severe or intolerable adverse effects. In some embodiments, the present invention provides an ophthalmic solution comprising about 0.1% to about 0.5% w/v reproxalap, or a pharmaceutically acceptable salt thereof, and a pharmaceutically acceptable excipient, wherein the molar ratio of reproxalap to the excipient is about 1:2.1 or less.

In some embodiments, the excipient is a cyclodextrin and the ratio of reproxalap to the excipient is about 1:2.1 to about 1:25.

In some embodiments, the present invention provides an ophthalmic solution comprising reproxalap, or a pharmaceutically acceptable salt thereof, and a pharmaceutically acceptable excipient, wherein the concentration of reproxalap, or a pharmaceutically acceptable salt thereof, is about 0.5% w/v or less and about 0.1% w/v or greater. In some embodiments, the ophthalmic solution comprises 0.15 to 0.45% w/v reproxalap, or a pharmaceutically acceptable salt thereof, and a pharmaceutically acceptable excipient. In some embodiments, the ophthalmic solution comprises about 0.2 to about 0.4% w/v reproxalap, or a pharmaceutically acceptable salt thereof, and a pharmaceutically acceptable excipient. In some embodiments, the ophthalmic solution comprises about 0.21 to about 0.35% w/v reproxalap, or a pharmaceutically acceptable salt thereof, and a pharmaceutically acceptable excipient. In some embodiments, the ophthalmic solution comprises about 0.22 to about 0.3% w/v reproxalap, or a pharmaceutically acceptable salt thereof, and a pharmaceutically acceptable excipient. In some embodiments, the ophthalmic solution comprises about 0.22 to about 0.29% w/v reproxalap, or a pharmaceutically acceptable salt thereof, and a pharmaceutically acceptable excipient. In some embodiments, the ophthalmic solution comprises about 0.25% w/v reproxalap, or a pharmaceutically acceptable salt thereof, and a pharmaceutically acceptable excipient. In some embodiments, the ophthalmic solution comprises about 0.25% w/v reproxalap and a pharmaceutically acceptable excipient selected from a cyclodextrin. In some embodiments, the ophthalmic solution comprises about 0.5% w/v reproxalap and a pharmaceutically acceptable excipient selected from a cyclodextrin.

In some embodiments, the present invention provides an ophthalmic solution comprising less than 0.5% w/v reproxalap, or a pharmaceutically acceptable salt thereof, and a pharmaceutically acceptable excipient. In some embodiments, the present invention provides an ophthalmic solution comprising at least 0.1% w/v reproxalap, or a pharmaceutically acceptable salt thereof, and a pharmaceutically acceptable excipient. In some embodiments, the present invention provides an ophthalmic solution comprising reproxalap, or a pharmaceutically acceptable salt thereof, and a pharmaceutically acceptable excipient, wherein the concentration of reproxalap, or a pharmaceutically acceptable salt thereof, is less than 0.5% w/v and 0.1% w/v or greater.

In some embodiments, reproxalap, or a pharmaceutically acceptable salt thereof, in an ophthalmic solution of the invention is at a concentration of less than 0.45% w/v and at least 0.1% w/v. In some embodiments, reproxalap, or a pharmaceutically acceptable salt thereof, in an ophthalmic solution of the invention is at a concentration of less than 0.4% w/v and at least 0.1% w/v. In some embodiments, reproxalap, or a pharmaceutically acceptable salt thereof, in an ophthalmic solution of the invention is at a concentration of less than 0.35% w/v and at least 0.1% w/v. In some embodiments, reproxalap, or a pharmaceutically acceptable salt thereof, in an ophthalmic solution of the invention is at a concentration of less than 0.3% w/v and at least 0.1% w/v. In some embodiments, reproxalap, or a pharmaceutically acceptable salt thereof, in an ophthalmic solution of the invention is at a concentration of less than 0.25% w/v and more than 0.1% w/v. In some embodiments, reproxalap, or a pharmaceutically acceptable salt thereof, in an ophthalmic solution of the invention is at a concentration of less than 0.2% w/v and at least 0.1% w/v. In some embodiments, reproxalap, or a pharmaceutically acceptable salt thereof, in an ophthalmic solution of the invention is at a concentration of less than 0.15% w/v and at least 0.1% w/v.

In some embodiments, reproxalap, or a pharmaceutically acceptable salt thereof, in an ophthalmic solution of the invention is at a concentration of 0.5% w/v or less and at least 0.15% w/v. In some embodiments, reproxalap, or a pharmaceutically acceptable salt thereof, in an ophthalmic solution of the invention is at a concentration of 0.5% w/v or less and at least 0.2% w/v. In some embodiments, reproxalap, or a pharmaceutically acceptable salt thereof, in an ophthalmic solution of the invention is at a concentration of 0.5% w/v or less and at least 0.25% w/v. In some embodiments, reproxalap, or a pharmaceutically acceptable salt thereof, in an ophthalmic solution of the invention is at a concentration of 0.5% w/v or less and at least 0.3% w/v. In some embodiments, reproxalap, or a pharmaceutically acceptable salt thereof, in an ophthalmic solution of the invention is at a concentration of 0.5% w/v or less and at least 0.35% w/v. In some embodiments, reproxalap, or a pharmaceutically acceptable salt thereof, in an ophthalmic solution of the invention is at a concentration of 0.5% w/v or less and at least 0.4% w/v. In some embodiments, reproxalap, or a pharmaceutically acceptable salt thereof, in an ophthalmic solution of the invention is at a concentration of 0.5% w/v or less and at least 0.45% w/v.

In some embodiments, reproxalap, or a pharmaceutically acceptable salt thereof, in an ophthalmic solution of the invention is at a concentration of about 0.1% to 0.5%, 0.15% to 0.45% w/v, 0.15% to 0.4% w/v, 0.15% to 0.35% w/v, 0.15% to 0.3% w/v, 0.15% to 0.25% w/v, or 0.15% to 0.2% w/v. In some embodiments, reproxalap, or a pharmaceutically acceptable salt thereof, in an ophthalmic solution of the invention is at a concentration of 0.2% to 0.45% w/v, 0.2% to 0.4% w/v, 0.2% to 0.35% w/v, 0.2% to 0.3% w/v, or 0.2% to 0.25% w/v. In some embodiments, reproxalap, or a pharmaceutically acceptable salt thereof, in an ophthalmic solution of the invention is at a concentration of 0.25% to 0.45% w/v, 0.25% to 0.4% w/v, 0.25% to 0.35% w/v, or 0.25% to 0.3% w/v. In some embodiments, reproxalap, or a pharmaceutically acceptable salt thereof, in an ophthalmic solution of the invention is at a concentration of 0.3% to 0.45% w/v or 0.3% to 0.4% w/v.

In some embodiments, reproxalap, or a pharmaceutically acceptable salt thereof, in an ophthalmic solution of the invention is at a concentration of about 0.1% w/v, 0.15% w/v, about 0.2% w/v, about 0.25%, about 0.3% w/v, about 0.35% w/v, about 0.4% w/v, about 0.45% w/v, or about 0.5% w/v.

In some embodiments, as further described herein, the foregoing concentrations of reproxalap can be selected and applied to treatment regimen that includes an initiation phase, an exacerbation phase, and/or a maintenance phase, as further described herein.

In some embodiments, a pharmaceutically acceptable excipient in an ophthalmic solution of the invention is a cyclodextrin. In some embodiments, a cyclodextrin is α-, β-, or γ-cyclodextrin. In some embodiments, a cyclodextrin is a pharmaceutically acceptable derivative of a cyclodextrin, including, but not limited to, the hydroxyalkyl derivatives of α-, β- and γ-cyclodextrin (especially the hydroxyethyl and hydroxypropyl derivatives of β-cyclodextrin and γ-cyclodextrin), randomly methylated β-cyclodextrin, sulfobutylether β-cyclodextrin, sulfobutylether γ-cyclodextrin, and the so-called branched β- and γ-cyclodextrin derivatives such as glucosyl-β-cyclodextrin and glucosyl-γ-cyclodextrin. The natural cyclodextrins are either used alone or in a mixture of two or more cyclodextrins, by way of non-limiting example, a mixture of the γ-cyclodextrin and the more water-soluble hydroxypropyl γ-cyclodextrin, or γ-cyclodextrin and sulfobutylether γ-cyclodextrin, or β-cyclodextrin and hydroxypropyl-β-cyclodextrin, or β-cyclodextrin and sulfobutylether β-cyclodextrin.

In some embodiments, a cyclodextrin in an ophthalmic solution of the invention is at a concentration of 0 to 20% w/v. In some embodiments, a cyclodextrin in an ophthalmic solution of the invention is at a concentration of 1 to 18% w/v, 1 to 16% w/v, 1 to 14% w/v, 2 to 12% w/v, 4 to 10% w/v, 5 to 9% w/v, or 6 to 8% w/v. In some embodiments, a cyclodextrin in an ophthalmic solution of the invention is at a concentration of about 1% w/v, 2% w/v, 3% w/v, 4% w/v, 5% w/v, 6% w/v, 7% w/v, 8% w/v, 9% w/v, 10% w/v, 11% w/v, 12% w/v, 13% w/v, 14% w/v, 15% w/v, 16% w/v, 17% w/v, 18% w/v, 19% w/v, or 20% w/v.

In some embodiments, a pharmaceutically acceptable excipient in an ophthalmic solution of the invention is sulfobutylether-β-cyclodextrin, in particular at any of the specified concentrations and ranges of concentrations above, such as about 7% w/v. In some embodiments, a pharmaceutically acceptable excipient in an ophthalmic solution of the invention is hydroxypropyl-β-cyclodextrin, in particular at any of the specified concentrations and ranges of concentrations specified above, such as about 7% w/v. In some embodiments, reproxalap is at a concentration of about 0.25% w/v, and the cyclodextrin, such as sulfobutylether-β-cyclodextrin or hydroxypropyl-β-cyclodextrin, is at a concentration of about 7% w/v.

In some embodiments, a pharmaceutically acceptable excipient in an ophthalmic solution of the invention is sulfobutylether-β-cyclodextrin, in particular at any of the specified concentrations and ranges of concentrations above, such as about 11% w/v. In some embodiments, a pharmaceutically acceptable excipient in an ophthalmic solution of the invention is hydroxypropyl-β-cyclodextrin, in particular at any of the specified concentrations and ranges of concentrations specified above, such as about 11% w/v. In some embodiments, reproxalap is at a concentration of about 0.25% w/v, and the cyclodextrin, such as sulfobutylether-β-cyclodextrin or hydroxypropyl-β-cyclodextrin, is at a concentration of about 11% w/v.

In some embodiments, an ophthalmic solution of the invention comprises a pharmaceutically acceptable buffering agent. In some embodiments, a pharmaceutically acceptable buffering agent is a phosphate buffer, citrate buffer, tris buffer, histidine buffer or acetate buffer.

In some embodiments, a pharmaceutically acceptable buffering agent is sodium phosphate, dibasic. In some embodiments, a pharmaceutically acceptable buffering agent is sodium phosphate, monobasic. In some embodiments, a pharmaceutically acceptable buffering agent is a mixture of sodium phosphate, dibasic, and sodium phosphate, monobasic. In some embodiments, an ophthalmic solution of the invention comprises about 0.083% w/v sodium phosphate, dibasic, and about 0.017% w/v sodium phosphate, monobasic.

In some embodiments, the ophthalmic solution of the invention is at an approximately neutral pH. In some embodiments, an ophthalmic solution of the invention is at a pH of 6.5 to 8. In some embodiments, an ophthalmic solution of the invention is at a pH of 6.9 to 7.7. In some embodiments, an ophthalmic solution of the invention is at a pH of 7.1 to 7.5. In some embodiments, an ophthalmic solution of the invention is at a pH of about 7.3.

Pharmaceutically acceptable acids and/or bases may be used in the ophthalmic solution to adjust pH. In some embodiments, an ophthalmic solution of the invention comprises a pharmaceutically acceptable acid. In some embodiments, an ophthalmic solution of the invention comprises a pharmaceutically acceptable base. In some embodiments, an ophthalmic solution of the invention comprises a pharmaceutically acceptable acid and base. In some embodiments, the pharmaceutically acceptable acid is hydrochloric acid. In some embodiments, the pharmaceutically acceptable base is sodium hydroxide.

In some embodiments, an ophthalmic solution of the invention comprises a tonicity agent. In some embodiments, a tonicity agent is selected from the group consisting of dextrose, potassium chloride, propylene glycol, and sodium chloride. In some embodiments, an ophthalmic solution of the invention comprises a tonicity agent at a concentration of less than about 0.5% w/v. In some embodiments, an ophthalmic solution of the invention comprises a tonicity agent at a concentration of about 0.45%, 0.4%, 0.35%, 0.3%, 0.25%, 0.2%, 0.15%, or 0.1% w/v. In some embodiments, a tonicity agent is sodium chloride.

In some embodiments, the ophthalmic solution comprises reproxalap at the specified concentrations, cyclodextrin, phosphate, and sodium chloride. In some embodiments, the ophthalmic solution comprises reproxalap at the specified concentrations herein (e.g., 0.1% w/v, 0.25% w/v, 0.5% w/v, etc.), 5 to 9% w/v cyclodextrin (e.g., sulfobutylether-β-cyclodextrin or hydroxypropyl-β-cyclodextrin); 0.07% to 0.09% w/v sodium phosphate (dibasic), 0.015% to 0.19% w/v sodium phosphate (monobasic), and 0.2 to 0.3% w/v sodium chloride. In some embodiments, the ophthalmic solution comprises reproxalap at the specified concentrations herein (e.g., 0.1% w/v, 0.25% w/v, 0.5% w/v, etc.), about 7% w/v cyclodextrin (e.g., sulfobutylether-β-cyclodextrin or hydroxypropyl-β-cyclodextrin); 0.07% to 0.09% w/v sodium phosphate (dibasic), 0.015% to 0.019% w/v sodium phosphate (monobasic), and 0.2 to 0.3% w/v sodium chloride. In some embodiments, the ophthalmic solution is adjusted to an appropriate pH with sodium hydroxide or HCl.

In some embodiments, the ophthalmic solution comprises reproxalap at the specified concentrations, cyclodextrin, phosphate, and sodium chloride. In some embodiments, the ophthalmic solution comprises reproxalap at the specified concentrations herein (e.g., 0.1% w/v, 0.25% w/v, 0.5% w/v, etc.), 10 to 14% w/v cyclodextrin (e.g., sulfobutylether-β-cyclodextrin or hydroxypropyl-β-cyclodextrin); 0.07% to 0.09% w/v sodium phosphate (dibasic), 0.015% to 0.19% w/v sodium phosphate (monobasic), and 0.2 to 0.3% w/v sodium chloride. In some embodiments, the ophthalmic solution comprises reproxalap at the specified concentrations herein (e.g., 0.1% w/v, 0.25% w/v, 0.5% w/v, etc.), about 11% w/v cyclodextrin (e.g., sulfobutylether-β-cyclodextrin or hydroxypropyl-β-cyclodextrin); 0.07% to 0.09% w/v sodium phosphate (dibasic), 0.015% to 0.019% w/v sodium phosphate (monobasic), and 0.2 to 0.3% w/v sodium chloride. In some embodiments, the ophthalmic solution is adjusted to an appropriate pH with sodium hydroxide or HCl.

In some embodiments, the ophthalmic solution comprises the composition of 0.5% Reproxalap Ophthalmic Solution A in the Examples. In some embodiments, the ophthalmic solution comprises the composition of 0.5% Reproxalap Ophthalmic Solution B in the Examples. In some embodiments, the ophthalmic solution comprises the composition of 0.25% Reproxalap Ophthalmic Solution A in the Examples. In some embodiments, the ophthalmic solution comprises the composition of 0.25% Reproxalap Ophthalmic Solution (Basic) in the Examples.

### 4. Methods of Treatment

In one aspect, the present invention provides a method for treating allergic conjunctivitis in a subject, comprising topically administering to an eye of a subject in need thereof an ophthalmic solution of the invention. In some embodiments, the concentration of reproxalap in the ophthalmic solution used in the method is as described above.

In some embodiments, an ophthalmic solution of the invention can be administered at different frequencies suitable for effectively treating allergic conjunctivitis, for example, without causing severe or intolerable adverse effects.

In some embodiments, the allergic conjunctivitis for treatment is seasonal allergic conjunctivitis. In some embodiments, the allergic conjunctivitis for treatment is contact allergic conjunctivitis. In some embodiments, the allergic conjunctivitis for treatment is perennial conjunctivitis.

In some embodiments, an ophthalmic solution of the invention can be topically administered one (1) to twelve (12) times a day onto the eye. In some embodiments, an ophthalmic solution of the invention can be topically administered one (1) to ten (10) times a day onto the eye. In some embodiments, an ophthalmic solution of the invention can be topically administered one (1) to eight (8) times a day onto the eye. In some embodiments, an ophthalmic solution of the invention can be topically administered one (1) to six (6) times a day onto the eye. In some embodiments, an ophthalmic solution of the invention can be topically administered one (1) to four (4) times a day onto the eye.

In some embodiments, a method of the invention comprises topically administering an ophthalmic solution of the invention up to twelve times a day. In some embodiments, a method of the invention comprises topically administering an ophthalmic solution of the invention up to eleven times a day. In some embodiments, a method of the invention comprises topically administering an ophthalmic solution of the invention up to ten times a day. In some embodiments, a method of the invention comprises topically administering an ophthalmic solution of the invention up to nine times a day. In some embodiments, a method of the invention comprises topically administering an ophthalmic solution of the invention up to eight times a day. In some embodiments, a method of the invention comprises topically administering an ophthalmic solution of the invention up to seven times a day. In some embodiments, a method of the invention comprises topically administering an ophthalmic solution of the invention six times a day. In some embodiments, a method of the invention comprises topically administering an ophthalmic solution of the invention five times a day. In some embodiments, a method of the invention comprises topically administering an ophthalmic solution of the invention four times a day (QID). In some embodiments, a method of the invention comprises topically administering an ophthalmic solution of the invention three times a day (TID). In some embodiments, a method of the invention comprises topically administering an ophthalmic solution of the invention two times a day (BID). In some embodiments, a method of the invention comprises topically administering an ophthalmic solution of the invention once a day (QD). In some embodiments, a method of the invention comprises topically administering an ophthalmic solution of the invention as needed (PRN).

In some embodiments, the present invention provides a method for treating allergic conjunctivitis in a subject, comprising topically administering to an eye of the subject an ophthalmic solution of the invention. In some embodiments, a method of the invention comprises topically administering an ophthalmic solution of the invention four times a day (QID). In some embodiments, a method of the invention comprises topically administering an ophthalmic solution of the invention three times a day (TID). In some embodiments, a method of the invention comprises topically administering an ophthalmic solution of the invention two times a day (BID). In some embodiments, a method of the invention comprises topically administering an ophthalmic solution of the invention once a day (QD). In some embodiments, a method of the invention comprises topically administering an ophthalmic solution of the invention as needed (PRN).

In some embodiments, a method of treating allergic conjunctivitis comprises topically administering to an eye of a subject in need thereof an ophthalmic solution of the invention four times a day (QID), three times a day (TID), two times a day (BID), once a day (QD), followed by administration as needed by the subject (PRN). In some embodiments, a method of treating allergic conjunctivitis comprises topically administering an ophthalmic solution of the invention four times a day (QID) followed by administration as needed (PRN).

In some embodiments, a method of treating allergic conjunctivitis comprises topically administering to an eye of a subject in need thereof an ophthalmic solution of the invention prior to an expected or certain exposure to an eye allergen. In some embodiments, the ophthalmic solution is topically administered to an eye within the time period of durability of the therapeutic effect, e.g., for a 0.25% w/v or 0.5% w/v reproxalap solution, prior to an expected or certain exposure to an eye allergen. In some embodiments, an ophthalmic solution of the invention is administered 3.5 h or less, 3 h or less, 2.5 h or less, 2 h or less, 1.5 h or less, 1 h or less, 0.5 h or less, 25 min or less, 20 min or less, 25 min or less, 20 min or less, 15 min or less, 10 min or less, or 5 min or less prior to an expected or certain exposure to an eye allergen, or immediately prior to exposure to an eye allergen. In some embodiments, an ophthalmic solution of the invention is administered 1.5 h or less, 1 h or less, 0.5 h or less, 25 min or less, 20 min or less, 25 min or less, 20 min or less, 15 min or less, 10 min or less, or 5 min or less prior to an expected or certain exposure to an eye allergen. In some embodiments, an ophthalmic solution of the invention is administered 0-0.5 h, 0-1 h, 0-1.5 h, 0-2 h, 0-2.5 h, 0-3 h, 0-3.5 h prior to an expected or certain exposure to an eye allergen. In some embodiments, an ophthalmic solution of the invention is administered 0.5-1 h, 0.5-1.5 h, 0.5-2 h, 0.5-2.5 h, 0.5-3 h, 0.5-3.5 h prior to an expected or certain exposure to an eye allergen. In some embodiments, an ophthalmic solution of the invention is administered 1-1.5 h, 1-2 h, 1-2.5 h, 1-3 h, or 1-3.5 h prior to an expected or certain exposure to an eye allergen. In some embodiments, an ophthalmic solution of the invention is administered 1.5-2 h, 1.5-2.5 h, 1.5-3 h, 1.5-3.5 h prior to an expected or certain exposure to an eye allergen. In some embodiments, an ophthalmic solution of the invention is administered −2.5 h, 2-3 h, 2-3.5 h prior to an expected or certain exposure to an eye allergen. In some embodiments, an ophthalmic solution of the invention is administered 2.5-3 h or 2.5-3.5 h prior to an expected or certain exposure to an eye allergen.

In some embodiments, a method for treating allergic conjunctivitis comprises topically administering to an eye of a subject in need thereof an ophthalmic solution of the invention one time, up to two times, up to three times, up to four times, up to five times, up to six times, up to seven times, or up to eight times prior to an expected or certain exposure to an eye allergen. In some embodiments, a method for treating allergic conjunctivitis comprises topically administering to an eye of a subject in need thereof an ophthalmic solution of the invention one time, two times, three times, four times, five times, six times, seven times, or eight times prior to an expected or certain exposure to an eye allergen. In some embodiments, the ophthalmic solution when administered two or more times is administered over 3.5 h, 3 h, 2.5 h, 2 h, 1.5 h, 1 h, or 0.5 h period before an expected or certain exposure to an eye allergen. In some embodiments, the ophthalmic solution when administered one time is administered 1.5 h or less, 1 h or less, 0.5 h or less, 25 min or less, 20 min or less, 25 min or less, 20 min or less, 15 min or less, 10 min or less, or 5 min or less prior to an expected or certain exposure to an eye allergen, or immediately prior to exposure to an expected or certain exposure to an eye allergen. In some embodiments, each time of administration of the ophthalmic solution is with one or two doses (e.g., drops) of the ophthalmic solution. In some embodiments, each time of administration of the ophthalmic solution is with a single dose (e.g., one drop) of the ophthalmic solution.

In some embodiments, the subject for treatment selected for treatment prior to an expected or certain exposure to an eye allergen has a history of allergic conjunctivitis. In some embodiments, the subject selected for treatment prior to an expected or certain exposure to an eye allergen has a history of allergic conjunctivitis and has a positive allergen skin test for one or more eye allergens, in particular for the eye allergen to which the subject is expected to be exposed. In some embodiments, the subject selected for treatment prior to an expected or certain exposure to an eye allergen has been previously clinically diagnosed with allergic conjunctivitis. In some embodiments, the subject selected for treatment prior to an expected or certain exposure to an eye allergen has been previously clinically diagnosed with allergic conjunctivitis and has a positive allergen skin test for one or more eye allergens, in particular for the eye allergen to which the subject is expected to be exposed. For the allergen skin test, a panel of allergens, particularly airborne allergens can be used, including allergens such as Hazel (e.g., Corylus avellana), Alder (e.g., Alnus incana), Birch (e.g., Betula alba), Plane (e.g., Platanus vulgaris), Cypress (e.g., Cupressus sempervirens), Grass mix (e.g., smooth meadow grass/Poa pratensis, cock's foot grass/Dactilis glomerata, perennial rye grass/Lolium perenne, timothy grass/Phleum pratense, meadow fescue/Festuca pratensis, meadow oat grass/Helictotrichon pretense), Olive (e.g., Olea europaea), Mugwort (e.g., Artemisia vulgaris), Ragweed (e.g., Ambrosia artemisiifolia), Alternaria (e.g., Alternaria alternata (tenuis)), Cladosporium (e.g., Cladosporium herbarum), Aspergillus (e.g., Aspergillus fumigatus), Parietaria (e.g., Parietaria), Cat, Dog, Dermatophagoides pteronyssinus, Dermatophagoides farina, and Blatella (e.g., Blatella germanica).

In some embodiments, a method for treating allergic conjunctivitis in a subject comprises administering to an eye of a subject in need thereof an ophthalmic solution of the invention at or after exposure to the eye allergen. In some embodiments, a method for treating allergic conjunctivitis in a subject comprises administering an ophthalmic solution of the invention to an eye of a subject in need thereof immediately after exposure to an eye allergen. In some embodiments, a method for treating allergic conjunctivitis comprises administering to an eye of a subject in need thereof an ophthalmic solution of the invention at or after the onset of symptoms of allergic conjunctivitis. In some embodiments, a method for treating allergic conjunctivitis comprises administering to an eye of a subject in need thereof an ophthalmic solution of the invention immediately after the onset of symptoms of allergic conjunctivitis. In some embodiments, a method for treating allergic conjunctivitis comprises administering to an eye of a subject in need thereof an ophthalmic solution of the invention prior to reaching highest severity of symptoms of allergic conjunctivitis. In some embodiments, the severity of symptoms of allergic conjunctivitis can use the scoring for ocular itch, conjunctival redness, and ocular tearing used in the Examples below or as is known in the art. In some embodiments, the scoring for severity of symptoms of allergic conjunctivitis are described in Ciolino et al., Clinical Ophthalmology, 2015, 9:765-772; Abelson et al., J Ocular Pharmacol., 1998, 14(6):533-542; and McLaurin et al., Clinical Sci., 2015, 34(10):1245-1251; all publications incorporated by reference herein). In some embodiments, the treatment at or after exposure to the eye allergen and/or after the onset of symptoms of allergic conjunctivitis can further include an initiation phase, exacerbation phase, and/or maintenance phase, as further discussed herein.

In some embodiments, a method for treating allergic conjunctivitis in a subject comprises administering to an eye of a subject in need thereof an ophthalmic solution of the invention by 5 min or less, 10 min or less, 15 min or less, 20 min or less, 25 min or less, 30 min or less, 35 min or less, 40 min or less, 45 min or less, 50 min or less, 60 min (1 h) or less, 75 min (1.25 h) or less, 90 min (1.5 h) or less, 105 min (1.75 h) or less, or 120 min (2 h) or less after exposure to an eye allergen or after the onset of symptoms of allergic conjunctivitis. In some embodiments, a method for treating allergic conjunctivitis in a subject comprises administering to an eye of a subject in need thereof an ophthalmic solution of the invention by 5 min or less, 10 min or less, 15 min or less, 20 min or less, 25 min or less, 30 min or less, 35 min or less, 40 min or less, 45 min or less, 50 min or less, 60 min (1 h) or less, 75 min (1.25 h) or less, or 90 min (1.5 h) or less after exposure to an eye allergen or after the onset of symptoms of allergic conjunctivitis. In some embodiments of the method, an ophthalmic solution of the invention is administered about 0-5 min, 0-10 min, 0-15 min, 0-20 min, 0-25 min, 0-30 min, 0-35 min, 0-40 min, 0-45 min, 0-50 min, 0-60 min, 0-75 min, 0-90 min, 0-105 min, or 0-120 min following exposure to an eye allergen or after the onset of symptoms of allergic conjunctivitis. In some embodiments of the method, an ophthalmic solution of the invention is administered about 5-10 min, 5-15 min, 5-20 min, 5-25 min, 5-30 min, 5-35 min, 5-40 min, 5-45 min, 5-50 min, 5-60 min, 5-75 min, 5-90 min, 5-105 min, or 5-120 min after exposure to an eye allergen or after the onset of symptoms of allergic conjunctivitis. In some embodiments of the method, an ophthalmic solution of the invention is administered about 10-15 min, 10-20 min, 10-25 min, 10-30 min, 10-35 min, 10-40 min, 10-45 min, 10-50 min, 10-60 min, 10-75 min, 10-90 min, 10-105 min, or 10-120 min after exposure to an eye allergen or after the onset of symptoms of allergic conjunctivitis. In some embodiments of the method, an ophthalmic solution of the invention is administered within 15-20 min, 15-25 min, 15-30 min, 15-35 min, 15-40 min, 15-45 min, 15-50 min, 15-60 min, 15-75 min, 15-90 min, 15-105 min, or 15-120 min after exposure to an eye allergen or after the onset of symptoms of allergic conjunctivitis. In some embodiments of the method, an ophthalmic solution of the invention is administered by 20-25 min, 20-30 min, 20-35 min, 20-40 min, 20-45 min, 20-50 min, 20-60 min, 20-75 min, 20-90 min, 20-105 min, or 20-120 min after exposure to an eye allergen or after the onset of symptoms of allergic conjunctivitis. In some embodiments of the method, an ophthalmic solution of the invention is administered by 25-30 min, 25-35 min, 25-40 min, 25-45 min, 25-50 min, 25-60 min, 25-75 min, 25-90 min, 25-105 min, or 25-120 min after exposure to an eye allergen or after the onset of symptoms of allergic conjunctivitis. In some embodiments of the method, an ophthalmic solution of the invention is administered by 30-35 min, 30-40 min, 30-45 min, 30-50 min, 30-60 min, 30-75 min, 30-90 min, 30-105 min, or 30-120 min after exposure to an eye allergen or after the onset of symptoms of allergic conjunctivitis. In some embodiments of the method, an ophthalmic solution of the invention is administered by 35-40 min, 35-45 min, 35-50 min, 35-60 min, 35-75 min, 35-90 min, 35-105 min, or 35-120 min after exposure to an eye allergen or after the onset of symptoms of allergic conjunctivitis. In some embodiments of the method, an ophthalmic solution of the invention is administered by 40-45 min, 40-50 min, 40-60 min, 40-75 min, 40-90 min, 40-105 min, or 40-120 min after exposure to an eye allergen or after the onset of symptoms of allergic conjunctivitis. In some embodiments of the method, an ophthalmic solution of the invention is administered by 45-50 min, 45-60 min, 45-75 min, 45-90 min, 45-105 min, or 45-120 min after exposure to an eye allergen or after the onset of symptoms of allergic conjunctivitis. In some embodiments of the method, an ophthalmic solution of the invention is administered by 50-60 min, 50-75 min, 50-90 min, 50-105 min, or 50-120 min after exposure to an eye allergen or after the onset of symptoms of allergic conjunctivitis. In some embodiments of the method, an ophthalmic solution of the invention is administered by 60-75 min, 60-90 min, 60-105 min, or 60-120 min after exposure to an eye allergen or after the onset of symptoms of allergic conjunctivitis. In some embodiments of the method, an ophthalmic solution of the invention is administered by 75-90 min, 75-105 min, or 75-120 min following exposure to an eye allergen or after the onset of symptoms of allergic conjunctivitis. In some embodiments of the method, an ophthalmic solution of the invention is administered by 90-105 min or 90-120 min after exposure to an eye allergen or after the onset of symptoms of allergic conjunctivitis.

In some embodiments, a method for treating allergic conjunctivitis in a subject comprises topically administering to an eye of a subject in need thereof an ophthalmic solution of the invention at or after exposure to an eye allergen, or at or after onset of symptoms of allergic conjunctivitis, where the ophthalmic solution is administered up to twelve times a day, up to eleven times a day, up to ten times a day, up to nine times a day, up to eight times a day, up to seven times a day, up to six times a day, up to five times a day, up to four times a day, up to three times a day, up to two times a day, or once a day. In some embodiments, a method for treating allergic conjunctivitis in a subject comprises topically administering to an eye of a subject in need thereof an ophthalmic solution of the invention at or after exposure to an eye allergen, or at or after onset of symptoms of allergic conjunctivitis, where the ophthalmic solution is administered twelve times a day, eleven times a day, ten times a day, nine times a day, eight times a day, seven times a day six times a day, five times a day, four times a day (QID), three times a day (TID), two times a day (BID), once a day (QD). In some embodiments, a method for treating allergic conjunctivitis in a subject comprises topically administering to any eye of a subject in need thereof an ophthalmic solution of the invention at or after exposure to an eye allergen, or after onset of symptoms of allergic conjunctivitis, where the ophthalmic solution is administered as needed (PRN). In some embodiments, a method for treating allergic conjunctivitis in a subject comprises topically administering to an eye of a subject in need thereof an ophthalmic solution of the invention at or after exposure to an eye allergen, or at or after onset of symptoms of allergic conjunctivitis, where the ophthalmic solution is administered four times a day (QID), three times a day (TID), two times a day (BID), once a day (QD), or as needed (PRN). In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where the ophthalmic solution is administered six times a day, five times a day, four times a day (QID), three times a day (TID), two times a day (BID), once a day (QD), followed by administration as needed (PRN). In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where the ophthalmic solution is administered four times a day (QID), three times a day (TID), two times a day (BID), once a day (QD), followed by administration as needed by the subject (PRN). In some embodiments, an ophthalmic solution of the invention is administered four times a day (QID) followed by administration as needed (PRN).

In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where each time of administration is every 1 h, 1.5 h, 2 h, 2.5 h, 3 h, 3.5 h, or 4 h. In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where each time of administration is every 1-1.5 h, 1-2 h, 1-2.5 h, 1-3 h, 1-3.5 h, or 1-4 h. In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where each time of administration is every 1.5-2 h, 1.5-2.5 h, 1.5-3 h, 1.5-3.5 h, or 1.5-4 h. In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where each time of administration is every 2-2.5 h, 2-3 h, 2-3.5 h, or 2-4 h. In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, where each time of administration is every 3-3.5 h or 3-4 h.

In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, wherein the ophthalmic solution is administered two (2) times a day (BID), and each time of administration is 60 min (1 h) apart; 90 min (1.5 h) apart; 120 min (2 h) apart; 150 min (2.5 h) apart; 180 min (3 h) apart; 210 min (3.5 h) apart; or 240 min (4 h) apart. In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, wherein the ophthalmic solution is administered three (3) times a day (TID) and each time of administration is 60 min (1 h) apart; 90 min (1.5 h) apart; 120 min (2 h) apart; 150 min (2.5 h) apart; 180 min (3 h) apart; 210 min (3.5 h) apart; or 240 min (4 h) apart. In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, wherein the ophthalmic solution is administered four (4) times a day (QID) and each time of administration is 60 min (1 h) apart; 90 min (1.5 h) apart; 120 min (2 h) apart; 150 min (2.5 h) apart; 180 min (3 h) apart; 210 min (3.5 h) apart; or 240 min (4 h) apart. In some embodiments, a method for treating allergic conjunctivitis comprises topically administering to an eye of a subject in need thereof an ophthalmic solution of the invention at or after exposure to an eye allergen, or at or after onset of symptoms of allergic conjunctivitis, wherein the ophthalmic solution is administered two (2) times a day (BID), and each time of administration is 60 min (1 h) apart; 90 min (1.5 h) apart; or 120 min (2 h) apart.

In some embodiments, each time of administration of the ophthalmic solution at or after exposure to an eye allergen, or at or after onset of symptoms of allergic conjunctivitis is with one or two doses (e.g., drops) of the ophthalmic solution. In some embodiments, each time of administration of the ophthalmic solution is with a single dose (e.g., one drop) of the ophthalmic solution. In some embodiments, each time of administration of the ophthalmic solution is with two doses (e.g., two drops) of the ophthalmic solution.

In some embodiments, the subject for treatment selected for treatment at or after exposure to an eye allergen, or at or after onset of symptoms of allergic conjunctivitis has a history of allergic conjunctivitis. In some embodiments, the subject selected for treatment at or after exposure to an eye allergen, or at or after onset of symptoms of allergic conjunctivitis has a history of allergic conjunctivitis and has a positive allergen skin test for one or more eye allergens, in particular for the eye allergen to which the subject is expected to be exposed. In some embodiments, the subject selected for treatment at or after exposure to an eye allergen, or at or after onset of symptoms of allergic conjunctivitis has been previously clinically diagnosed with allergic conjunctivitis. In some embodiments, the subject selected for treatment at or after exposure to an eye allergen, or at or after onset of symptoms of allergic conjunctivitis has been previously clinically diagnosed with allergic conjunctivitis and has a positive allergen skin test for one or more eye allergens, in particular for the eye allergen to which the subject is expected to be exposed. In some embodiments, a standard allergen skin test employs a panel of allergens, such as that described above.

In some embodiments, a method for treating allergic conjunctivitis comprises administering to an eye of a subject in need thereof an ophthalmic solution of the invention prior to an expected or certain exposure to an eye allergen, and further comprising administering an ophthalmic solution of the invention at or after exposure to the eye allergen, or at or after onset of symptoms of allergic conjunctivitis. This treatment protocol provides a two phased treatment regimen, the first phase being administration prior to an expected or certain exposure to an eye allergen, and a second phase being administration at or after exposure of an eye allergen, or at or after the onset of symptoms of eye allergy. In some embodiments, a method for treating allergic conjunctivitis comprises a first treatment phase comprising administering the ophthalmic solution prior to an expected or certain exposure of the subject to an eye allergen; and a second treatment phase comprising administering the ophthalmic solution at or after exposure of the subject to an eye allergen, or at or after onset of symptoms of allergic conjunctivitis. In some embodiments, the second phase of treatment can further include treatment in an initiation phase, exacerbation phase, and/or maintenance phase, as discussed herein.

In some embodiments, in the first treatment phase (administering to an eye of a subject in need thereof an ophthalmic solution of the invention prior to an expected or certain exposure to an eye allergen), an ophthalmic solution is topically administered to an eye within the time period of durability of the therapeutic effect, e.g., for a 0.25% w/v or 0.5% w/v reproxalap solution, prior to an expected or certain exposure to an eye allergen. In some embodiments, in the first treatment phase, an ophthalmic solution of the invention is administered 3.5 h or less, 3 h or less, 2.5 h or less, 2 h or less, 1.5 h or less, 1 h or less, 0.5 h or less, 25 min or less, 20 min or less, 25 min or less, 20 min or less, 15 min or less, 10 min or less, or 5 min or less prior to an expected or certain exposure to an eye allergen, or immediately prior to exposure to an eye allergen. In some embodiments, an ophthalmic solution of the invention is administered 0-0.5 h, 0-1 h, 0-1.5 h, 0-2 h, 0-2.5 h, 0-3 h, 0-3.5 h prior to an expected or certain exposure to an eye allergen. In some embodiments, an ophthalmic solution of the invention is administered 0.5-1 h, 0.5-1.5 h, 0.5-2 h, 0.5-2.5 h, 0.5-3 h, 0.5-3.5 h prior to an expected or certain exposure to an eye allergen. In some embodiments, an ophthalmic solution of the invention is administered 1-1.5 h, 1-2 h, 1-2.5 h, 1-3 h, 1-3.5 h prior to an expected or certain exposure to an eye allergen. In some embodiments, an ophthalmic solution of the invention is administered 1.5-2 h, 1.5-2.5 h, 1.5-3 h, 1.5-3.5 h prior to an expected or certain exposure to an eye allergen. In some embodiments, an ophthalmic solution of the invention is administered 2-2.5 h, 2-3 h, 2-3.5 h prior to an expected or certain exposure to an eye allergen. In some embodiments, an ophthalmic solution of the invention is administered 2.5-3 h or 2.5-3.5 h prior to an expected or certain exposure to an eye allergen.

In some embodiments, in the first treatment phase (administering to an eye of a subject in need thereof an ophthalmic solution of the invention prior to an expected or certain exposure to an eye allergen), an ophthalmic solution of the invention is administered one time, up to two times, up to three times, up to four times, up to five times, up to six times, up to seven times, or up to eight times prior to an expected or certain exposure to an eye allergen. In some embodiments, in the first treatment phase, an ophthalmic solution of the invention is administered one time, two times, three times, four times, five times, six times, seven times, or eight times prior to an expected or certain exposure to an eye allergen. In some embodiments, in the first treatment phase, an ophthalmic solution of the invention is administered one time, up to two times, up to three times, or up to four times prior to an expected or certain exposure to an eye allergen. In some embodiments, in the first treatment phase, an ophthalmic solution of the invention is administered one time, two times, three times, or four times prior to an expected or certain exposure to an eye allergen. In some embodiments, the ophthalmic composition when administered two or more times is administered over 3.5 h, 3 h, 2.5 h, 2 h, 1.5 h, 1 h, or 0.5 h before an expected or certain exposure to an eye allergen. In some embodiments of the first treatment phase, the ophthalmic solution when administered one time is administered 1.5 h or less, 1 h or less, 0.5 h or less, 25 min or less, 20 min or less, 25 min or less, 20 min or less, 15 min or less, 10 min or less, or 5 min or less prior to an expected or certain exposure to an eye allergen, or immediately prior to exposure to an expected or certain exposure to an eye allergen. In some embodiments, each time of administration of the ophthalmic solution in the first treatment phase is with one or two doses (e.g., drops) of the ophthalmic solution. In some embodiments, each time of administration of the ophthalmic solution in the first treatment phase is with a single dose (e.g., one drop) of the ophthalmic solution). In some embodiments, each time of administration of the ophthalmic solution in the first treatment phase is with two doses (e.g., two drops) of the ophthalmic solution.

In some embodiments, in the second treatment phase (administering to an eye of a subject in need thereof an ophthalmic solution of the invention in the period at or after exposure to an eye allergen, or at or after onset of symptoms of allergic conjunctivitis), an ophthalmic solution of the invention is administered within 5 min, 10 min, 15 min, 20 min, 25 min, 30 min, 35 min, 40 min, 45 min, 50 min, 60 min (1 h), 75 min (1.25 h), 90 min (1.5 h), 105 min (1.75 h), or 120 min (2 h) after the exposure to an eye allergen or after the onset of symptoms of allergic conjunctivitis. In some embodiments, in the second treatment phase, an ophthalmic solution of the invention is administered within 0-5 min, 0-10 min, 0-15 min, 0-20 min, 0-25 min, 0-30 min, 0-35 min, 0-40 min, 0-45 min, 0-50 min, 0-60 min, 0-75 min, 0-90 min, 0-105 min, or 0-120 min following exposure to an eye allergen or after the onset of symptoms of allergic conjunctivitis. In some embodiments of the method, an ophthalmic solution of the invention is administered within 5-10 min, 5-15 min, 5-20 min, 5-25 min, 5-30 min, 5-35 min, 5-40 min, 5-45 min, 5-50 min, 5-60 min, 5-75 min, 5-90 min, 5-105 min, or 5-120 min following exposure to an eye allergen or after the onset of symptoms of allergic conjunctivitis. In some embodiments of the method, an ophthalmic solution of the invention is administered within 10-15 min, 10-20 min, 10-25 min, 10-30 min, 10-35 min, 10-40 min, 10-45 min, 10-50 min, 10-60 min, 10-75 min, 10-90 min, 10-105 min, or 10-120 min after exposure to an eye allergen or after the onset of symptoms of allergic conjunctivitis. In some embodiments of the method, an ophthalmic solution of the invention is administered within 15-20 min, 15-25 min, 15-30 min, 15-35 min, 15-40 min, 15-45 min, 15-50 min, 15-60 min, 15-75 min, 15-90 min, 15-105 min, or 15-120 min following exposure to an eye allergen or after the onset of symptoms of allergic conjunctivitis. In some embodiments of the method, an ophthalmic solution of the invention is administered within 20-25 min, 20-30 min, 20-35 min, 20-40 min, 20-45 min, 20-50 min, 20-60 min, 20-75 min, 20-90 min, 20-105 min, or 20-120 min following exposure to an eye allergen or after the onset of symptoms of allergic conjunctivitis. In some embodiments of the method, an ophthalmic solution of the invention is administered within 25-30 min, 25-35 min, 25-40 min, 25-45 min, 25-50 min, 25-60 min, 25-75 min, 25-90 min, 25-105 min, or 25-120 min following exposure to an eye allergen or after the onset of symptoms of allergic conjunctivitis. In some embodiments of the method, an ophthalmic solution of the invention is administered within 30-35 min, 30-40 min, 30-45 min, 30-50 min, 30-60 min, 30-75 min, 30-90 min, 30-105 min, or 30-120 min following exposure to an eye allergen or after the onset of symptoms of allergic conjunctivitis. In some embodiments of the method, an ophthalmic solution of the invention is administered within 35-40 min, 35-45 min, 35-50 min, 35-60 min, 35-75 min, 35-90 min, 35-105 min, or 35-120 min following exposure to an eye allergen or after the onset of symptoms of allergic conjunctivitis. In some embodiments of the method, an ophthalmic solution of the invention is administered within 40-45 min, 40-50 min, 40-60 min, 40-75 min, 40-90 min, 40-105 min, or 40-120 min following exposure to an eye allergen or after the onset of symptoms of allergic conjunctivitis. In some embodiments of the method, an ophthalmic solution of the invention is administered within 45-50 min, 45-60 min, 45-75 min, 45-90 min, 45-105 min, or 45-120 min following exposure to an eye allergen or after the onset of symptoms of allergic conjunctivitis. In some embodiments of the method, an ophthalmic solution of the invention is administered within 50-60 min, 50-75 min, 50-90 min, 50-105 min, or 50-120 min following exposure to an eye allergen or after the onset of symptoms of allergic conjunctivitis. In some embodiments of the method, an ophthalmic solution of the invention is administered within 60-75 min, 60-90 min, 60-105 min, or 60-120 min following exposure to an eye allergen or after the onset of symptoms of allergic conjunctivitis. In some embodiments of the method, an ophthalmic solution of the invention is administered within 75-90 min, 75-105 min, or 75-120 min following exposure to an eye allergen or after the onset of symptoms of allergic conjunctivitis. In some embodiments of the method, an ophthalmic solution of the invention is administered within 90-105 min, or 90-120 min following exposure to an eye allergen or after the onset of symptoms of allergic conjunctivitis.

In some embodiments, in the second treatment phase, an ophthalmic solution of the invention is administered up to eight times a day, up to seven times a day, up to six times a day, up to five times a day, up to four times a day, up to three times a day, up to two times a day, or once a day. In some embodiments, in the second treatment phase, an ophthalmic solution of the invention is administered eight times a day, seven times a day, six times a day, five times a day, four times a day (QID), three times a day (TID), two times a day (BID), or once a day (QD). In some embodiments, in the second treatment phase, an ophthalmic solution of the invention is administered as needed (PRN). In some embodiments, in the second treatment phase, an ophthalmic solution of the invention is administered eight times a day, seven times a day, six times a day, five times a day, four times a day (QID), three times a day (TID), two times a day (BID), once a day (QD), followed by administration as needed (PRN). In some embodiments of the method, an ophthalmic solution of the invention is administered four times a day (QID) followed by administration as needed (PRN).

In some embodiments, in the second treatment phase, each time of administration is every 1 h, 1.5 h, 2 h, 2.5 h, 3 h, 3.5 h, or 4 h. In some embodiments of the method each time of administration is every 1-1.5 h, 1-2 h, 1-2.5 h, 1-3 h, 1-3.5 h, or 1-4 h. In some embodiments of the method, each time of administration is every 1.5-2 h, 1.5-2.5 h, 1.5-3 h, 1.5-3.5 h, or 1.5-4 h. In some embodiments of the method, each time of administration is every 2-2.5 h, 2-3 h, 2-3.5 h, or 2-4 h. In some embodiments of the method, each time of administration is every 3-3.5 h or 3-4 h.

In some embodiments, in the second treatment phase, an ophthalmic solution of the invention is administered two (2) times a day (BID), and each time of administration is 60 min (1 h) apart; 90 min (1.5 h) apart; 120 min (2 h) apart; 150 min (2.5 h) apart; 180 min (3 h) apart; 210 min (3.5 h) apart; or 240 min (4 h) apart. In some embodiments of the method, an ophthalmic solution of the invention is administered three (3) times a day (TID) and each time of administration is 60 min (1 h) apart; 90 min (1.5 h) apart; 120 min (2 h) apart; 150 min (2.5 h) apart; 180 min (3 h) apart; 210 min (3.5 h) apart; or 240 min (4 h) apart. In some embodiments of the method, an ophthalmic solution of the invention is administered four (4) times a day (QID) and each time of administration is 60 min (1 h) apart; 90 min (1.5 h) apart; 120 min (2 h) apart; 150 min (2.5 h) apart; 180 min (3 h) apart; 210 min (3.5 h) apart; or 240 min (4 h) apart. In some embodiments of the method, an ophthalmic solution of the invention is administered two (2) times a day (BID), and each time of administration is 60 min (1 h) apart; 90 min (1.5 h) apart; or 120 min (2 h) apart.

In some embodiments, in the second treatment phase, each time of administration of the ophthalmic solution at or after exposure to an eye allergen, or at or after onset of symptoms of allergic conjunctivitis is with one or two doses (e.g., drops) of the ophthalmic solution. In some embodiments, in the second treatment phase, each time of administration of the ophthalmic solution is with a single dose (e.g., one drop) of the ophthalmic solution. In some embodiments, in the second treatment phase, each time of administration of the ophthalmic solution is with two doses (e.g., two drops) of the ophthalmic solution.

In some embodiments, in the method for treating allergic conjunctivitis comprising a first treatment phase and a second treatment phase, wherein the first treatment phase comprises administering the ophthalmic solution prior to an expected or certain exposure of the subject to an eye allergen; and the second treatment phase comprises administering the ophthalmic solution at or after exposure of the subject to an eye allergen, or at or after onset of symptoms of allergic conjunctivitis, the subject selected for treatment has a history of allergic conjunctivitis. In some embodiments, the subject selected for treatment has a history of allergic conjunctivitis and has a positive allergen skin test for one or more eye allergens, in particular for the eye allergen to which the subject is expected to be exposed. In some embodiments, the subject selected for treatment has been previously clinically diagnosed with allergic conjunctivitis. In some embodiments, the subject selected for treatment has been previously clinically diagnosed with allergic conjunctivitis and has a positive allergen skin test for one or more eye allergens, in particular for the eye allergen to which the subject is expected to be exposed. In some embodiments, a standard allergen skin test employs a panel of allergens, such as that described above.

In some embodiments, a method of the invention comprises topically administering an ophthalmic solution of the invention at various strengths (for example, at different reproxalap concentrations and different administration frequencies, as described herein).

In some embodiments, a method of the invention comprises topically administering an ophthalmic solution comprising about 0.25% w/v reproxalap, or a pharmaceutically acceptable salt thereof, four times a day, three times a day, or two times a day.

In some embodiments, a method of the invention comprises topically administering an ophthalmic solution comprising about 0.30% w/v reproxalap, or a pharmaceutically acceptable salt thereof, four times a day, three times a day, or two times a day.

In some embodiments, a method of the invention comprises topically administering an ophthalmic solution comprising about 0.35% w/v reproxalap, or a pharmaceutically acceptable salt thereof, four times a day, three times a day, or two times a day.

In some embodiments, a method of the invention comprises topically administering an ophthalmic solution comprising about 0.4% w/v reproxalap, or a pharmaceutically acceptable salt thereof, four times a day, three times a day, or two times a day.

In some embodiments, a method of the invention comprises topically administering an ophthalmic solution comprising about 0.45% w/v reproxalap, or a pharmaceutically acceptable salt thereof, four times a day, three times a day, or two times a day.

In some embodiments, a method of the invention comprises topically administering an ophthalmic solution comprising about 0.5% w/v reproxalap, or a pharmaceutically acceptable salt thereof, four times a day, three times a day, or two times a day.

In some embodiments, a method of the invention comprises topically administering an ophthalmic solution comprising 0.3% to 0.4% w/v reproxalap, or a pharmaceutically acceptable salt thereof, four times a day, three times a day, or two times a day.

In some embodiments, a method of the invention comprises topically administering an ophthalmic solution comprising 0.2% to 0.4% w/v reproxalap, or a pharmaceutically acceptable salt thereof, four times a day, three times a day, or two times a day.

In some embodiments, a method of the invention comprises treatment at two or more phases, including an initiation phase, exacerbation phase, and/or maintenance phase. In some embodiments, the treatments at the different phases can include administration of different strengths of reproxalap, different frequency of administration, or a combination of different strengths of reproxalap and different frequencies of administration. In some embodiments, the initiation phase of treatment is started at the onset of symptoms of allergic conjunctivitis. In some embodiments, the initiation phase of treatment is started when allergic conjunctivitis symptoms are at exacerbation of disease signs and/or symptoms. In some embodiments, the treatment in the initiation phase and/or exacerbation phase can be up to 1 week, up to 2 weeks, up to 3 weeks, up to 4 weeks, up to 6 weeks, up to 8 weeks, or up to 12 weeks. In some embodiments, the maintenance phase can be up to 1 week, up to 2 weeks, up to 3 weeks, up to 4 weeks, up to 6 weeks, up to 8 weeks, or up to 12 weeks or more, e.g., until symptoms of allergic conjunctivitis lessen or are resolved.

In some embodiments, a method of the invention comprises two or more phases, wherein an ophthalmic solution of the invention is topically administering at different strengths in different phases. In some embodiments, a method of the invention comprises an initiation phase and a maintenance phase, wherein the ophthalmic solution is topically administered at a higher strength in the initiation phase than in the maintenance phase. In some embodiments, a treatment cycle of a method of the invention comprising multiple phases, including an exacerbation phase during which signs and/or symptoms become worse.

In some embodiments, the method of the invention comprises two or more phases, wherein an ophthalmic solution of the invention is topically administering at different strengths in different phases. In some embodiments, a method of the invention comprises an initiation phase, wherein the ophthalmic solution is topically administered at a high strength in the initiation phase, at a low strength in the maintenance phase, and at a high strength during an exacerbation of disease signs and/or symptoms.

In some embodiments, an ophthalmic solution administered in an initiation phase comprises a higher concentration of reproxalap, or a pharmaceutically acceptable salt thereof, than an ophthalmic solution administered in a maintenance phase. In some embodiments, the ophthalmic solution administered in an initiation phase or an exacerbation phase and the ophthalmic solution administered in a maintenance phase, comprises reproxalap, or a pharmaceutically acceptable salt, at a concentration selected from the group consisting of about 0.5% w/v, 0.45% w/v, 0.4% w/v, 0.35% w/v, 0.3% w/v, 0.25% w/v, 0.2% w/v, 0.15% w/v, and 0.1% w/v.

In In some embodiments, an ophthalmic solution of about 0.5% w/v reproxalap is administered in an initiation phase or exacerbation phase, and less than 0.5% w/v reproxalap administered in a maintenance phase. In some embodiments, an ophthalmic solution of about 0.4% w/v, 0.35% w/v, 0.3% w/v, 0.25% w/v, 0.2% w/v, 0.15% w/v or 0.1% w/v reproxalap is administered in the maintenance phase.

In some embodiments, an ophthalmic solution of about 0.5% w/v to about 0.4% reproxalap is administered in an initiation phase or exacerbation phase, and less than 0.4% w/v reproxalap administered in a maintenance phase. In some embodiments, an ophthalmic solution of about 0.35% w/v, 0.3% w/v, 0.25% w/v, 0.2% w/v, 0.15% w/v or 0.1% w/v reproxalap is administered in the maintenance phase.

In some embodiments, an ophthalmic solution of about 0.5% w/v to about 0.3% reproxalap is administered in an initiation phase or exacerbation phase, and less than 0.3% w/v reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of about 0.4% w/v to about 0.3% reproxalap is administered in an initiation phase or exacerbation phase, and less than 0.3% w/v reproxalap administered in a maintenance phase. In some embodiments, an ophthalmic solution of about 0.25% w/v, 0.2% w/v, 0.15% w/v or 0.1% w/v reproxalap is administered in the maintenance phase

In some embodiments, an ophthalmic solution of about 0.3% w/v to about 0.2% reproxalap (e.g., 0.3%, 0.25%, or 0.2% w/v) is administered in an initiation phase or exacerbation phase, and 0.25% w/v or less reproxalap administered in a maintenance phase. In some embodiments, an ophthalmic solution of about 0.25% w/v, 0.2% w/v, 0.15% w/v or 0.1% w/v reproxalap is administered in the maintenance phase.

In some embodiments, an ophthalmic solution of the invention is topically administered more frequently per day in an initiation phase and/or an exacerbation phase than in a maintenance phase. In some embodiments, an ophthalmic solution of the invention is topically administered five times a day in an initiation phase, followed by four, three, two, one times a day, or as needed in a maintenance phase. In some embodiments, an ophthalmic solution of the invention is topically administered four times a day (QID) in an initiation phase or exacerbation phase, followed by three times a day (TID), two times a day (BID), once a day (QD), or as needed (PRN) in a maintenance phase. In some embodiments, an ophthalmic solution of the invention is topically administered three times a day (TID) in an initiation phase or exacerbation phase, followed by two times a day (BID) or once per day (QD), or as needed (PRN) in a maintenance phase. In some embodiments, an ophthalmic solution of the invention is topically administered two times a day (BID) in an initiation phase or exacerbation phase, followed by once daily (QD) or as needed (PRN) in a maintenance phase.

In some embodiments, an ophthalmic solution of the invention used for the initiation phase and the maintenance phase has the same concentration of reproxalap (e.g., 0.5%, 0.4% 0.3%, 0.25%, or 0.2% w/v) when the frequency of administration is varied for the initiation phase and the maintenance phase.

In some embodiments, an ophthalmic solution administered in an initiation phase or exacerbation phase is at a higher reproxalap concentration and higher administration frequency than an ophthalmic solution administered in a maintenance phase.

In some embodiments, the present invention provides a method for treating allergic conjunctivitis in a subject, comprising topically administering to the subject an ophthalmic solution comprising about 0.4% w/v reproxalap, or a pharmaceutically acceptable salt thereof, and a pharmaceutically acceptable excipient, wherein the ophthalmic solution is administered at a higher strength in an initiation phase or exacerbation phase followed by a lower strength in a maintenance phase, wherein each of the initiation phase, exacerbation phase, and maintenance phase is as described herein.

In some embodiments, a multiple phase treatment cycle can include an initiation phase or exacerbation phase of up to 1, up to 2, up to 3, up to 4 up to 6, up to 8, or up to 12 weeks with an ophthalmic solution comprising about 0.5%, 0.4% or 0.35% w/v, 0.25% (e.g., 0.5% to 0.35% w/v) reproxalap, or a pharmaceutically acceptable salt thereof, followed by a maintenance phase. In some embodiments, an ophthalmic solution comprising about 0.5%, 0.4% or 0.35% w/v, 0.25% (e.g., 0.5% to 0.35% w/v) reproxalap, or a pharmaceutically acceptable salt thereof, is administered four times a day (QID) in an initiation phase or exacerbation phase followed by three times a day (TID), two times a day (BID), or once per day (QD) in the maintenance phase. In some embodiments, an ophthalmic solution comprising about 0.5%, 0.4% or 0.35% w/v, 0.25% (e.g., 0.5% to 0.35% w/v) reproxalap, or a pharmaceutically acceptable salt thereof, is administered three times a day (TID) in an initiation phase or exacerbation phase followed by two times a day (BID) or once per day (QD) in the maintenance phase.

In some embodiments, an ophthalmic solution comprising about 0.4%, 0.35% or 0.3% w/v (e.g., 0.4% to 0.3% w/v) reproxalap, or a pharmaceutically acceptable salt thereof, is administered four times a day (QID) in an initiation phase or exacerbation phase followed by three times a day (TID), two times a day (BID), or once per day (QD) in the maintenance phase. In some embodiments, an ophthalmic solution comprising about 0.4%, 0.35% or 0.3% w/v (e.g., 0.4% to 0.3% w/v) reproxalap, or a pharmaceutically acceptable salt thereof, is administered three times a day (TID) in an initiation phase or exacerbation phase followed by two times a day (BID) or once per day (QD) in the maintenance phase.

In some embodiments, an ophthalmic solution comprising about 0.3%, 0.25% or 0.2% w/v (e.g., 0.3% to 0.2% w/v) reproxalap, or a pharmaceutically acceptable salt thereof, is administered four times a day (QID) in an initiation phase or exacerbation phase followed by three times a day (TID), two times a day (BID), or once per day (QD) in the maintenance phase. In some embodiments, an ophthalmic solution comprising about 0.3%, 0.25% or 0.2% w/v (e.g., 0.3% to 0.2% w/v) reproxalap, or a pharmaceutically acceptable salt thereof, is administered three times a day (TID) in an initiation phase or exacerbation phase followed by two times a day (BID) or once per day (QD) in the maintenance phase.

In some embodiments, the present invention provides a method for treating allergic conjunctivitis in a subject, comprising topically administering to the subject an ophthalmic solution comprising 0.35% to 0.45% w/v reproxalap, or a pharmaceutically acceptable salt thereof, and a pharmaceutically acceptable excipient, wherein the ophthalmic solution is administered at a higher strength in an initiation phase or exacerbation phase followed by a lower strength in a maintenance phase, wherein each of the initiation phase, exacerbation phase and maintenance phase is as described herein. In some embodiments, a multiple phase treatment cycle of an ophthalmic solution comprising 0.35% to 0.45% w/v reproxalap, or a pharmaceutically acceptable salt thereof, is up to 12 weeks. In some embodiments, an ophthalmic solution comprising 0.35% to 0.45% w/v reproxalap, or a pharmaceutically acceptable salt thereof, is administered four times a day (QID) in an initiation phase or exacerbation phase followed by three times a day (TID), two times a day (BID), or once per day (QD) in maintenance phase. In some embodiments, an ophthalmic solution comprising 0.35%-0.45% w/v reproxalap, or a pharmaceutically acceptable salt thereof, is administered three times a day (TID) in an initiation phase or exacerbation phase followed by two times a day (BID) or once per day (QD) in maintenance phase.

In some embodiments, the treatment regimen with an ophthalmic solution of the invention provides a statistically significant reduction in the ocular itch scale of a patient population suffering from allergic conjunctivitis. In some embodiments, the treatment regimen with the ophthalmic solution of the invention provides a statistically significant (p<0.05) reduction by at least 1 point in the ocular itch scale of a patient population suffering from allergic conjunctivitis. In some embodiments, the treatment regimen with the ophthalmic solution of the invention provides a statistically significant (p<0.05) reduction by at least 2 points in the ocular itch scale of a patient population suffering from allergic conjunctivitis. Ocular Itch Scale 0 (no itch) to 4 (incapacitating itch).

In some embodiments, the present invention provides a method for treating certain subjects with allergic conjunctivitis. In some embodiments, a subject with allergic conjunctivitis is 18 years or older. In some embodiments, a subject with allergic conjunctivitis has a history of allergic conjunctivitis for at least six months prior to receiving the treatment of the invention. In some embodiments, a subject with allergic conjunctivitis has a history of use or desire to use eye drops for symptoms within six months prior to receiving the treatment of the invention.

In some embodiments, the present invention provides a method for treating a subject with allergic conjunctivitis, comprising identifying subjects satisfying one or more of the following criteria for at least one eye, prior to receiving the treatment of the invention (for example, a screening performed at about one and/or two weeks before receiving the treatment):
having a Schirmer's Test score of ≤10 mm and ≥1 mm;
having a tear film break-up time (TFBUT®)≤5 seconds;
having a corneal fluorescein staining score of ≥2 in at least one region (e.g., inferior, superior, or central);
having a sum corneal fluorescein staining score of ≥4 based on the sum of the inferior, superior, and central regions; and
having a total Lissamine green conjunctival score of ≥2 based on the sum of the temporal and nasal regions.

In some embodiments, the present invention provides a method for treating a subject with allergic conjunctivitis comprising a screening to exclude subjects having one or more of the following conditions for at least one eye, prior to receiving the treatment of the invention:
having any clinically significant slit lamp findings that may include active blepharitis, meibomian gland dysfunction (MGD), lid margin inflammation, or active ocular allergies that may require therapeutic treatment;
having an ongoing ocular infection (bacterial, viral, or fungal), or active ocular inflammation;
having previously had laser-assisted in situ keratomileusis (LASIK) surgery within the last 12 months;
having any planned ocular and/or lid surgeries over the study period or any ocular surgery within six months; and
having a known allergy and/or sensitivity to an ophthalmic solution of the invention or its components.

As described herein, an ophthalmic solution of the invention can achieve an increased efficacy in subjects with allergic conjunctivitis. Accordingly, in some embodiments, the present invention provides a method for treating a subject with allergic conjunctivitis comprising topically administering to the subject an ophthalmic solution of the invention.

In some embodiments, the present invention provides a method for treating allergic conjunctivitis in a subject, comprising topically administering to the subject an ophthalmic solution described herein, wherein the ophthalmic solution is administered three times a day (TID), two times a day (BID), or once per day (QD). In some embodiments, an ophthalmic solution comprising about 0.25% w/v reproxalap, or a pharmaceutically acceptable salt thereof, is administered three times a day (TID). In some embodiments, an ophthalmic solution comprising about 0.25% w/v reproxalap, or a pharmaceutically acceptable salt thereof, is administered two times a day (BID). In some embodiments, an ophthalmic solution comprising about 0.25% w/v reproxalap, or a pharmaceutically acceptable salt thereof, is administered once daily (QD).

For topical ophthalmic administration, each administration comprises one or more aliquots of the composition (e.g., ophthalmic solution). In some embodiments, the aliquot administered in an estimated volume, for example an applied drop using a dropper or a squeeze vial. In some embodiments, one or more drops, two drops, three drops, up to 4 drops are topically applied to an eye or to each of both eyes at each administration. In some embodiments, each administration comprises sequential administration, for example, a first administration of one or more aliquots (e.g., one or more drops), a first time period for allowing absorption of the composition, followed by a second administration of one or more aliquots (e.g., one or more drops). In some embodiments, one drop provides a single dose of the ophthalmic solution. In some embodiments, the volume for topical administration is about 25 uL to about 75 uL. In some embodiment, the volume for topical administration is about 30 uL to about 60 uL. In some embodiments, the volume for topical administration is about 50 uL. In some embodiments, the volume of one drop of an ophthalmic solution of the invention is approximately 30 uL to 50 uL. In some embodiments, the volume of one drop of an ophthalmic solution of the invention is approximately 50 uL.

In some embodiments, a presently disclosed ophthalmic solution is co-administered with an additional therapeutic agent. In some embodiments, the additional therapeutic agent is an agent disclosed in WO2017147617 or WO2017196881, each of which is hereby incorporated by reference in its entirety. In some embodiments, the additional therapeutic agent is an antihistamine, mast cell inhibitor, or anti-inflammatory agent. Exemplary antihistamines include, among others, levocabastine, olopatadine, and ketotifen fumarate. In some embodiments, the anti-inflammatory agent is a steroid, non-steroidal anti-inflammatory compound, anti-metabolite, immunosuppressive antibiotic, alkylating agent, or an anti-inflammatory cytokine antibody. Exemplary steroidal compounds include, among others, cortisol, cortisone, prednisone, prednisolone (e.g., prednisolone acetate), methylprednisone, triamcinolone, betamethasone, dexamethasone and a prodrug thereof. Exemplary non-steroidal anti-inflammatory compounds include, among others, acetylsalicylic acid, diflunisal, salsalate, ibuprofen, dexibuprofen, naioxen, fenoprofen, ketoprofen, dexketoprofen, flurbiprofen, oxaprozin, loxoprofen, indomethacin, tolmetin, sulindac, etodolac, ketorolac, diclofenac, aceclofenac, nabumetone, piroxicam, meloxicam, tenoxicam, droxicam, lornoxicam, phenylbu alone, mefenamic acid, meclofenamic acid, flufenamic acid, tolfenamic acid, and celecoxib. Exemplary antimetabolites include, among others, azathioprine, mercaptopurine, leflunomide, mycophenolic acid, and mycophenolate mofetil, and methotrexate. Exemplary anti-inflammatory antibiotics include, among others, cyclosporine, tacrolimus, rapamycin, and derivatives thereof. Exemplary anti-inflammatory antibodies, include among others, antibodies targeting an inflammatory cytokine, such as antibodies against TNFα. IL-1, IL-4, IL-5, and IL-17.

In another aspect, further provided herein are methods for assessing the safety, efficacy, comparability, or bioequivalence of an aldehyde trapping compound for treating allergic conjunctivitis using an EEC chamber protocol. In some embodiments, a method for assessing the safety, efficacy, comparability or bioequivalence of an aldehyde trapping compound for treating allergic conjunctivitis, comprising placing a test subject in an EEC chamber, providing one or more test allergens, administering a test aldehyde trapping compound or a composition thereof, and assessing severity of symptoms of allergic conjunctivitis. In some embodiments, the symptoms for assessing severity of allergic conjunctivitis includes, among others, one or more of ocular itch, ocular tearing, and conjunctival redness. In some embodiments, the test aldehyde trapping compound or a composition thereof is administered prior to and/or at start of or after exposure of the test subjects to the allergen. In some embodiments, the method is used to assess the comparability or bioequivalence of ophthalmic compositions containing 0.25% w/v or 0.5% w/v of reproxalap.

### EXAMPLES

The following examples are intended to illustrate the invention and are not to be construed as being limitations thereon. Reproxalap can be synthesized as reported previously, for example, in WO 2006/127945, the entire content of which is incorporated herein by reference.

Abbreviations
CAE: controlled adverse environment
GMP: Good Manufacturing Practice
ICH: International Council for Harmonization of Technical Requirements for Pharmaceuticals for Human Use
OD: right eye
OS: left eye
OU: both eyes
PRN: as needed
QD: once daily
QID: Four times daily
QS: as much as will suffice

### Example 1. A Multi-Center, Double-Masked, Randomized, Parallel-Group, Vehicle-Controlled, Phase 3 Clinical Trial to Assess the Safety and Efficacy of Reproxalap Ophthalmic Solutions (0.25% and 0.5%) Compared to Vehicle in the Conjunctival Allergen Challenge (Ora-CAC®) Model of Acute Allergic Conjunctivitis

A double-masked, randomized, vehicle-controlled, multi-center, parallel-group conjunctival allergen challenge Phase 3 trial assessed the efficacy and safety of 0.25% and 0.5% concentrations of reproxalap topical ophthalmic solutions compared to vehicle in 318 patients (approximately 100 per arm) with seasonal allergic conjunctivitis. The primary efficacy endpoint was the evaluation of ocular itch score (0 to 4 scale) area under the curve from 10 to 60 minutes after allergen challenge, and the key secondary endpoint was two-point responder rate, a measure of clinical relevance.

The primary objective for the study was statistically significant improvement vs. vehicle (p<0.0001 and p=0.0025, respectively) on primary endpoint of ocular itch score area under the curve from 10-60 minutes after allergen challenge.

The secondary objective for the study was statistically significant improvement vs. vehicle (p=0.0005 and p=0.0169, respectively) on key secondary responder analysis of two-point improvement in ocular itch score (0-4 scale).

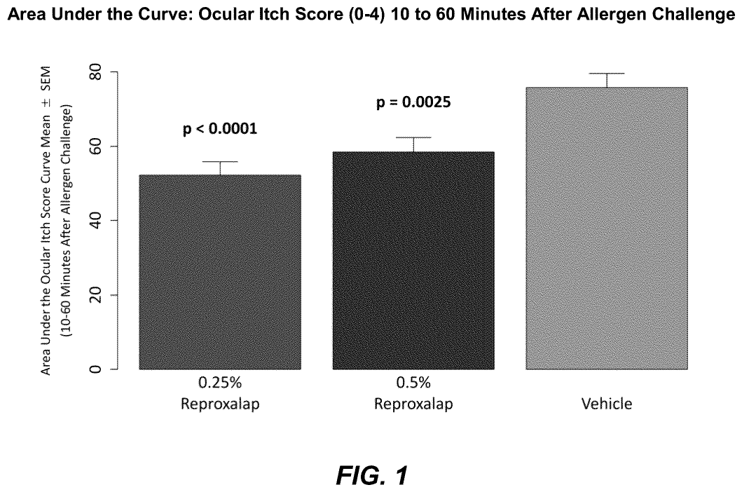

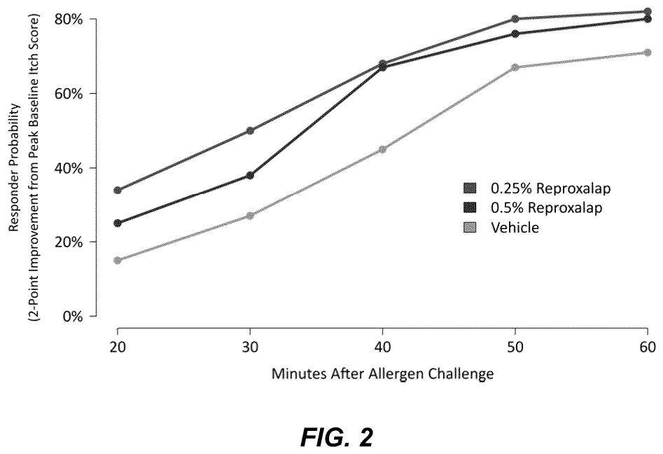

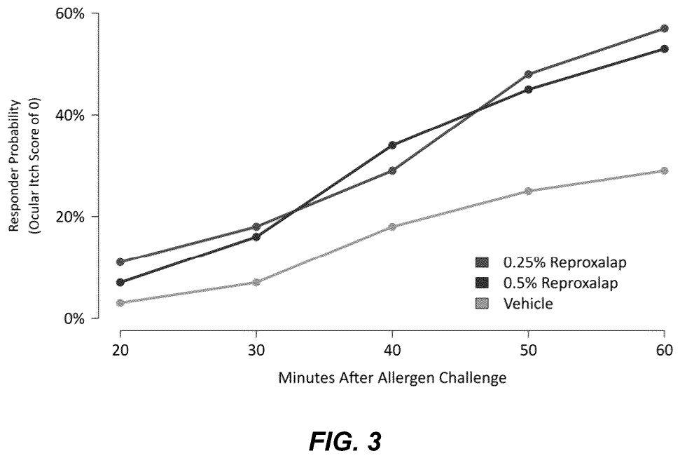

Relative to patients treated with vehicle, patients treated with 0.25% and 0.5% reproxalap demonstrated statistically significant reduction in ocular itching (p<0.0001 and p=0.0025, respectively; FIG. 1), as assessed by area under the ocular itch score curve. Two-point responder rates for 0.25% and 0.5% reproxalap were statistically greater than that of vehicle-treated patients (p=0.0005 and p=0.0169, respectively; FIGS. 2 and 3), confirming the clinical relevance of the observed primary endpoint improvements. Both concentrations of reproxalap exhibited an anti-inflammatory profile that is distinct from standard-of-care antihistamine therapy and supports a differentiated mechanism of action for the treatment of allergic conjunctivitis.

No adverse events were observed other than mild, transient instillation site irritation, consistent with prior reproxalap clinical trials.

No observed findings were observed on the following safety assessments: visual acuity (ETDRS chart), intraocular pressure (contact tonometry), slit lamp biomicroscopy, and dilated fundoscopy.

Duration

This trial consisted of four (4) office visits (Screening Visit, Visit 1, Visit 2, and Visit 3) over a period of approximately three to nine (3-9) weeks.

Screening Period: Screening was approximately 3-9 weeks in duration. At Visit 1, subjects underwent an ocular allergen challenge titration using an allergen that elicited a positive reaction via skin testing. Subjects with a positive reaction post-CAC underwent a confirmation CAC at Visit 2 with the same allergen qualified during Visit 1.

Treatment Period: Treatment begins at Visit 3 after subjects are randomized. At this visit, subjects receive an in-office dose of the treatment.

Inclusion Criteria

Each subject must:
be at least 18 years of age of either gender and any race;
have a positive history of ocular allergies and a positive skin test reaction to a seasonal allergen (grasses, ragweed, and/or trees) as confirmed by an allergic skin test at the Screening Visit or within the past 24 months;
be able and willing to avoid all disallowed medications for the appropriate washout period and during the trial;
be able and willing to discontinue wearing contact lenses for at least 72 hours prior to Visit 1 and during the trial period;
have a calculated visual acuity of 0.7 logMAR or better in each eye as measured using an ETDRS chart;
agree to have urine pregnancy testing (for women considered capable of becoming pregnant, including all females who have experienced menarche and have not experienced menopause [as defined by amenorrhea for greater than 12 consecutive months] or have not undergone successful surgical sterilization [hysterectomy, bilateral tubal ligation, or bilateral oophorectomy]) performed at screening and exit visit; not be lactating; and agree to use a medically acceptable form of birth control throughout the trial duration;
have a positive bilateral CAC reaction (≥2.5 ocular itching and ≥2 conjunctival redness) within 10 (±2) minutes of instillation of the last titration of allergen at Visit 1; and
have a positive bilateral CAC reaction (≥2.5 ocular itching and ≥2 conjunctival redness) for at least two out of the first three time points following the challenge at Visit 2.

Exclusion Criteria

Each subject may not:
have known contraindications or sensitivities to the use of the investigational product or any of its components;
have any ocular condition that, in the opinion of the investigator, could affect the subject's safety or trial parameters (including but not limited to narrow angle glaucoma, clinically significant blepharitis, follicular conjunctivitis, iritis, pterygium or a diagnosis of dry eye);
have had ocular surgical intervention within three (3) months prior to Visit 1 or during the trial and/or a history of refractive surgery six (6) months prior to Visit 1 or have ocular or systemic surgery planned during the study or within 30 days after;
have a known history of retinal detachment, diabetic retinopathy, or active retinal disease;
have the presence of an active ocular infection (bacterial, viral or fungal), any active uveitis, or positive history of ocular herpes or an ocular herpetic infection at any visit;
use any of the following disallowed medications during the period indicated prior to Visit 1 and during the trial:
   72 hours
      systemic or ocular H₁ antihistamine, H₁ antihistamine/mast cell stabilizers, H₁ antihistamine-vasoconstrictor drug combinations
   7 days
      decongestants
      monoamine oxidase inhibitors
      all other topical ophthalmic preparations (including artificial tears)
      lid scrubs
      topical prostaglandins or prostaglandin derivatives
      ocular, topical, or systemic nonsteroidal anti-inflammatory drugs (NSAIDs); Baby aspirin (81 mg) is allowed as long as a stable dose has been maintained for at least 30 days prior to Visit 1 and will continue to be maintained for the duration of the trial.
   14 days
      inhaled, ocular, topical, or systemic corticosteroids or mast cell stabilizers
   45 days
      depo-corticosteroids
   2 months
      immunotherapeutic agents (treatment must have been maintained steadily for at least 2 months; neither the immunotherapeutic agent nor its dosage may change during the clinical trial) (Note: Currently marketed over-the-counter anti-allergy eye drops (e.g., anti-histamine/vasoconstrictor combination products) may be administered by trained trial personnel to subjects at the end of Visits 1, 2, and 3, after all evaluations are completed.)
      have any significant illness (including, but not limited to, poorly controlled hypertension, poorly controlled diabetes, a history of status asthmaticus, a history of organ transplantation, a history of persistent moderate or severe asthma, a history of moderate to severe allergic asthmatic reactions to any of the trial allergens, any autoimmune disease requiring therapy, or severe cardiovascular disease or arrhythmia) that, at the investigators' discretion, could be expected to interfere with the subject's health or with the trial parameters and/or put the subject at any unnecessary risk;
      manifest signs or symptoms of clinically active allergic conjunctivitis in either eye at the start of Visits 1, 2, or 3 (defined as the presence of any itching or >1 [greater than 1] redness in the conjunctival vessel bed);
      have a history of glaucoma, ocular hypertension or an intraocular pressure (TOP) that is greater than 24 mmHg at Visit 1;
      have used an investigational drug or medical device within 30 days of the trial or be concurrently enrolled in another investigational product trial;
      be a female who is currently pregnant, planning a pregnancy, lactating, not using a medically acceptable form of birth control throughout the trial duration, or has a positive urine pregnancy test at Visit 1.

Statistical Analysis

The primary efficacy variable of ocular itch score (scale 0 to 4) area under the curve from 10-60 minutes post-challenge (AUC 10-60) is measured at Visit 3. A generalized linear model containing treatment as a main effect and, if useful, Visit 2 baseline information as one or more covariates, was fit to Visit 3 AUC 10-60 data. The model was used to evaluate two contrasts: a) Reproxalap Ophthalmic Solution 0.5% mean AUC 10-60 vs. Vehicle Ophthalmic Solution mean AUC 10-60, and b) Reproxalap Ophthalmic Solution 0.25% mean AUC 10-60 vs. Vehicle Ophthalmic Solution mean AUC 10-60. The primary efficacy analyses was conducted on the intent-to-treat (ITT) population.

The Key Secondary Endpoint (intended to be used for US NDA approval) of within-subject two-point response (i.e., two-point reduction from Visit 2 5-minute post-CAC itch score at 20 through 60 minutes post-CAC Visit 3) was evaluated using a GEE containing treatment, post-CAC Visit 3 measurement time, the treatment x time interaction, and, if useful, Visit 2 baseline information as one or more covariates. The model was used to evaluate the odds ratio of Reproxalap Ophthalmic Solution 0.5% vs. Vehicle Ophthalmic Solution, and Reproxalap Ophthalmic Solution 0.25% vs. Vehicle Ophthalmic Solution across combined Visit 3 post-CAC time points for the ITT population utilizing an assumed covariance matrix within the GEE to accommodate missing data. Significance levels for these contrasts was adjusted for multiplicity using the Hochberg procedure to avoid false discovery (Reference: FDA, Multiple Endpoints in Clinical Trials, Draft Guidance for Industry, January 2017).

Phase 3 AC Formulation: 0.5% Reproxalap Ophthalmic Solution A

| | Amount | |
|---|---|---|
| Component | (% w/v) | Grade |
| ADX-102 (reproxalap) | 0.5% | GMP |
| Sulfobutylether-beta-cyclodextrin | 9.5% | USP |
| (SBECD) | | |
| Sodium phosphate, dibasic, anhydrous | 0.083% | USP |
| Sodium phosphate, monobasic, | 0.017% | USP |
| monohydrate | | |
| Sodium hydroxide or | pH adjustment | USP/NF |
| Hydrochloric acid | | |
| Sterile Water for Injection (WFI) | Dilute to volume | USP |

Phase 3 AC Formulation: 0.5% Reproxalap Ophthalmic Solution B

| | Amount | |
|---|---|---|
| Component | (% w/v) | Grade |
| ADX-102 (reproxalap) | 0.5% | GMP |
| Sulfobutylether-beta-cyclodextrin | 9.5% | USP |
| (SBECD) | | |
| Sodium hydroxide or | pH adjustment | USP/NF |
| Hydrochloric acid | | |
| Sterile Water for Injection (WFI) | Dilute to volume | USP |

Phase 3 AC Formulation: 0.25% Reproxalap Ophthalmic Solution A

| | Amount | |
|---|---|---|
| Component | (% w/v) | Grade |
| ADX-102 (reproxalap) | 0.25% | GMP |
| Sulfobutylether-beta-cyclodextrin | 7.0% | USP |
| (SBECD) | | |
| Sodium phosphate, dibasic, anhydrous | 0.083% | USP |
| Sodium phosphate, monobasic, | 0.017% | USP |
| monohydrate | | |
| Sodium chloride | 0.24% | USP |
| Sodium chloride | Tonicity adjustment | USP |
| Sodium hydroxide or | pH adjustment | USP/NF |
| Hydrochloric acid | | |
| Sterile Water for Injection (WFI) | Dilute to volume | USP |

Phase 3 AC Formulation: 0.25% Reproxalap Ophthalmic Solution (Basic)

| | Amount | |
|---|---|---|
| Component | (% w/v) | Grade |
| ADX-102 (reproxalap) | 0.25% | GMP |
| Sulfobutylether-beta-cyclodextrin | 7.0% | USP |
| (SBECD) | | |
| Sodium chloride | Tonicity adjustment | USP |
| Sodium hydroxide or | pH adjustment | USP/NF |
| Hydrochloric acid | | |
| Sterile Water for Injection (WFI) | Dilute to volume | USP |

In some embodiments, a basic ophthalmic solution is provided above. Additional excipients may be optionally added to the basic ophthalmic solution.

Phase 3 AC Composition of Vehicle Ophthalmic Solution

| | Amount | |
|---|---|---|
| Component | (% w/v) | Grade |
| Sodium phosphate, dibasic, anhydrous | 0.083% | USP |
| Sodium phosphate, monobasic, | 0.017% | USP |
| monohydrate | | |
| Sodium Chloride | 0.9% | USP |
| Sodium hydroxide or | pH adjustment | USP/NF |
| Hydrochloric acid | | |
| Sterile Water for Injection (WFI) | Dilute to volume | USP |

Generally, the vehicle used is the same as the AC formulations but without the reproxalap. In some embodiments, where the AC formulation do not include sodium phosphate and/or sodium chloride, the vehicle formulation do not include the sodium phosphate and/or the sodium chloride.

### Example 2: A Clinical Trial Evaluating Reproxalap Ophthalmic Solutions in Subjects with Seasonal Allergic Conjunctivitis Using the Environmental Exposure Chamber (EEC)

The clinical trial evaluated the feasibility of using the Environmental Exposure Chamber (EEC) to assess the activity of Reproxalap Ophthalmic Solutions (0.25% and 0.5%) in an allergic conjunctivitis population.

The study assessed the safety, tolerability, and pharmacodynamic activity of Reproxalap ophthalmic solutions (0.25% and 0.5%) compared to vehicle ophthalmic solution in the treatment of seasonal allergic conjunctivitis in subjects allergic to ragweed using the EEC with exposure to airborne ragweed pollen. The EEC is a chamber, e.g., a room, that provides reproducible challenges with controlled levels with airborne allergen and control of environmental factors, such as humidity and temperature (Devillier et al., Allergy, 2011, 66(2):163-9; Goldstein et al., Eye & Contact Lens, 2015, 41(3):145-155; Jacobs et al., J Allergy Clin Immunol., 2012, 130:122-7). The EEC chambers have been used for Phase 3 studies to provide real-life conditions for natural allergen exposure (Pfaar et al., Allergy, 2017, 73(Suppl 104):5-23). It allows for better assessment of the dose response, onset of action, and magnitude of treatment effects, and further provides low variability in outcomes and reduced confounding factors−further controlled in this study with restrictions in timing for EEC assessments relative to work schedule

Inclusion/Exclusion Criteria included, among others, the following:
A history of moderate-to-severe ragweed-induced allergic conjunctivitis
Positive skin prick test to ragweed pollen within the past year of the Medical Screening Visit
Have a positive bilateral CAC reaction to ragweed allergen (≥2 ocular itching and redness scores) within 10 minutes of instillation of the last titration of allergen at Visit 2
Have signs and symptoms of allergic conjunctivitis in both eyes (≥1 ocular itching and redness) for ≥4 days of the 1 week artificial tears run-in period prior to Visit 3.

The EEC clinical research facility was designed with the capacity and control mechanisms to expose participants to airborne ragweed pollen grains and maintains target temperature and relative humidity throughout. The EEC has 100% fresh HEPA-filtered clean air to which commercially obtained air dried, non-defatted short ragweed (Ambrosia artemesiifolia) pollen (Greer Laboratories, Lenoir, NC) were introduced and circulated throughout the room with tight humidity and temperature controls as described previously (Ronborg et al., Allergy, 1996, 51(2):82-88). Airborne ragweed pollen counts were maintained at 3500±500 grains per cubic meter by continuous monitoring and feedback regulation of the pollen emission measured. The controlled pollen challenge, over time, allowed evaluation of subject responses at any time-point throughout the challenge process.

Patients were subject to medical screening and EEC screening, as highlighted above for inclusion/exclusion criteria. A skin prick test for a standard panel of test allergens was conducted including cat, dust, ragweed, grass or tree pollen. Results must be positive (i.e., a wheal 3 mm greater than the negative control) for at least one test allergen and must include ragweed in order to proceed to the Screening EEC Visit.

For EEC treatment sessions, patients were randomized into three treatment groups: (A) treatment with Reprolaxap 0.25% ophthalmic solution, (B) treatment with 0.5% ophthalmic solution, and (C) vehicle treatment. In the EEC treatment sessions, dosing was 5 min prior to entry into the chamber, and 95 min post entry into the chamber. The patients were in the EEC for approximately 215 min. Following exit from the EEC, patients were monitored for ocular itching and tearing for up to about 60 min. Staff assessed conjunctival redness were assessed up to about 60 min following exit from the chamber. The patients were subject to three sessions in the EEC chamber, where each EEC sessions were separated by approximately a two-week washout period to ensure adequate elimination of responses caused by allergen exposure in the EEC and allow the recuperation of mast cells.

For subject-reported symptoms of ocular itching, a standard 9 point (i.e., 0-4 with 0.5 unit increments) and a standard VAS scale (0-100 mm) were employed. For subject rated ocular tearing, a 4-point scale (0-3) was employed. For staff-assessed grading of conjunctival redness, a standard 9-point (i.e., 0-4 with 0.5 unit increments) was employed.

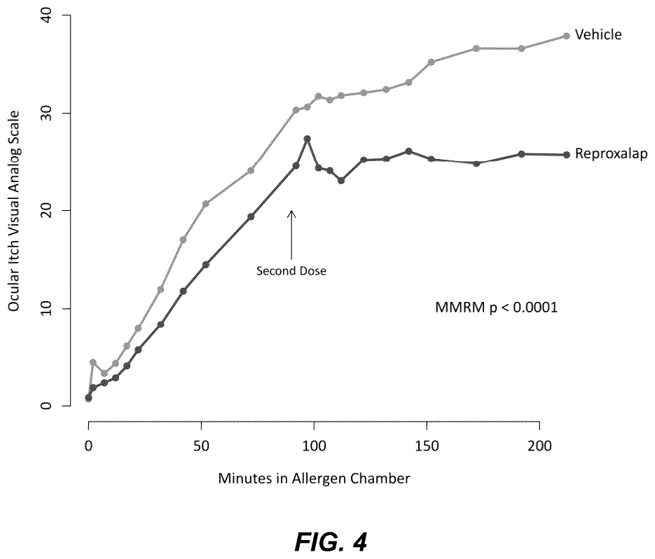

FIG. 4 shows the ocular itch score (0-100) during 200 minutes of allergen exposure in the EEC. Treatment with 0.25% w/v Reproxalap shows statistically significant reduction in ocular itch vs. vehicle for more than 3 hrs of exposure to allergen.

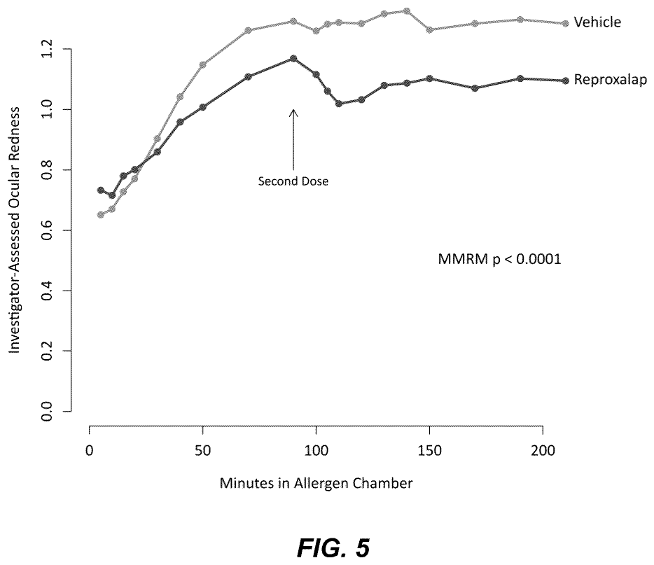

FIG. 5 shows the ocular redness score (0-4) during 200 minutes of allergen exposure in the EEC. Treatment with 0.25% w/v Reproxalap shows statistically significant reduction in ocular redness vs. vehicle for more than 3 hrs of exposure to allergen.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

All publications, patents, patent applications and other documents cited in this application are hereby incorporated by reference in their entireties for all purposes to the same extent as if each individual publication, patent, patent application or other document were individually indicated to be incorporated by reference for all purposes.

We claim:

1. A method of treating allergic conjunctivitis in a subject, comprising topically administering to an eye of a subject in need thereof a therapeutically effective amount of an ophthalmic solution comprising reproxalap, or a pharmaceutically acceptable salt thereof, and a pharmaceutically acceptable excipient, wherein the concentration of reproxalap, or a pharmaceutically acceptable salt thereof, is about 0.25% w/v, and the pharmaceutically acceptable excipient is about 11% w/v of sulfobutylether β-cyclodextrin (SBECD), or a pharmaceutically acceptable salt thereof.

2-9. (canceled)

10. The method of claim 1, wherein the ophthalmic solution further comprises a tonicity agent and a phosphate buffer.

11. The method of claim 1, wherein the administering is prior to an expected or certain exposure of the subject to an eye allergen, wherein the administering is 3.5 h or less, 3 h or less, 2.5 h or less, 2 h or less, 1.5 h or less, 1 h or less, 0.5 h or less, 25 min or less, 20 min or less, 25 min or less, 20 min or less, 15 min or less, 10 min or less, or 5 min or less prior to the expected or certain exposure to the eye allergen.

12-14. (canceled)

15. The method of claim 11, wherein the ophthalmic solution is administered one time, up to two times, up to three times, up to four times, up to five times, up to six times, up to seven times, or up to eight times prior to the expected or certain exposure to an eye allergen.

16. (canceled)

17. The method of claim 11, wherein the subject for treatment has a history of being afflicted with allergic conjunctivitis and/or has a positive skin test for one or more eye allergens.

18-20. (canceled)

21. The method of claim 1, wherein the administering is at or after exposure of the subject to an eye allergen, or at or after onset of symptoms of allergic conjunctivitis.

22-24. (canceled)

25. The method of claim 21, wherein the ophthalmic solution is administered up to twelve times a day, up to ten times a day, up to eight times a day, up to six times a day, up to five times a day, up to four times a day (QID), up to three times a day (TID), up to two times a day (BID), once a day (QD), or as needed (PRN) at or after exposure of the subject to an eye allergen, or at or after onset of symptoms of allergic conjunctivitis.

26. The method of claim 25, wherein the ophthalmic solution is administered twelve times a day, ten times a day, eight times a day, six times a day, five times a day, four times a day (QID), three times a day (TID), two times a day (BID), once a day (QD), or as needed (PRN).

27. The method of claim 21, wherein the ophthalmic solution is administered twelve times a day, ten times a day, eight times a day, six times a day, four times a day (QID), three times a day (TID), two times a day (BID), or once a day (QD), followed by administration as needed (PRN).

28-29. (canceled)

30. The method of claim 1, wherein the treating comprises: a first treatment phase comprising administering the ophthalmic solution prior to an expected or certain exposure of the subject to an eye allergen; and a second treatment phase comprising administering the ophthalmic solution at or after exposure of the subject to an eye allergen, or at or after onset of symptoms of allergic conjunctivitis.

31. The method of claim 30, wherein the first treatment phase comprises administering the ophthalmic solution 3.5 h or less, 3 h or less, 2.5 h or less, 2 h or less, 1.5 h or less, 1 h or less, 0.5 h or less, 25 min or less, 20 min or less, 25 min or less, 20 min or less, 15 min or less, 10 min or less, or 5 min or less prior to the expected or certain exposure to the eye allergen.

32-33. (canceled)

34. The method of claim 30, wherein the first treatment phase comprises administering one time, two times, three times, or four times prior to the expected or certain exposure to an eye allergen.

35-36. (canceled)

37. The method of claim 30, wherein the second treatment phase is at or after onset of symptoms of allergic conjunctivitis.

38. (canceled)

39. The method of claim 30, wherein the second treatment phase comprises administering the ophthalmic solution eight times a day, seven times a day, six times a day, five times a day, four times a day (QID), three times a day (TID), two times a day (BID), once a day (QD), or as needed (PRN).

40. The method of claim 30, wherein the second treatment phase comprises administering the ophthalmic solution eight times a day, seven times a day, six times a day, five times a day, four times a day (QID), three times a day (TID), two times a day (BID), once a day (QD), followed by administration as needed (PRN).

41. The method of claim 1, wherein the treatment includes an initiation phase, an exacerbation phase and/or a maintenance phase.

42. The method of claim 41, wherein the initiation phase or exacerbation phrase comprises administering the ophthalmic solution five or four times a day (QID).

43. The method of claim 41, wherein the maintenance phase comprises administering the ophthalmic solution four times a day (QID), three times a day (TID), two times a day (BID), once per day (QD) or as needed (PRN).

44.-46. (canceled)

47. The method of claim 1, wherein after administration to the subject, the ophthalmic solution provides a statistically significant reduction in the ocular itch scale of the patient as compared to no treatment or vehicle treatment.

48. The method of claim 47, wherein the reduction in the ocular itch score is by at least 1 point.

49-50. (canceled)
